(19) United States
(12) Patent Application Publication
(10) Pub. No.: US 2020/0289890 A1
(43) Pub. Date: Sep. 17, 2020

(54) Intelligent Exercise or Therapy Apparatus and Method

(71) Applicants: Alexander Kim, Valencia, CA (US); Sahm Bazargan, Simi Valley, CA (US)

(72) Inventors: Alexander Kim, Valencia, CA (US); Sahm Bazargan, Simi Valley, CA (US)

(21) Appl. No.: 16/799,791

(22) Filed: Feb. 24, 2020

Related U.S. Application Data
(60) Provisional application No. 62/814,714, filed on Mar. 6, 2019.

(51) Int. Cl. A63B 24/00; G16H 20/30; G16H 40/67; G16H 15/00; G16H 50/30; G01L 1/22; G01P 15/18; G01R 33/02; G01P 3/44; G01C 19/00; A63B 71/06; A63B 21/00; A63B 21/055; A63B 21/04; A63B 21/072

(52) U.S. Cl. CPC A63B 24/0062; G16H 10/60; G16H 40/67; G16H 15/00; G16H 50/30; G01L 1/22; G01P 15/18; G01R 33/0206; G01P 3/44; G01C 19/00; A63B 71/0622; A63B 21/4035; A63B 21/0552; A63B 21/0414; A63B 21/0724; G16H 20/30

(57) ABSTRACT

An intelligent exercise or therapy device one or more handles. The one or more handles include one or more accelerometers, one or more gyroscopes or directional sensors, one or more magnetometers, to capture multi-axis angular measurements, the one or more accelerometers to capture velocity or acceleration measurements; one or more pressure sensors or strain gauges to capture grip strength measurements; and one or more wireless communication transceivers to transmit the captured multi-axis measurements, the captured grip strength measurements and the captured velocity or acceleration measurements to a computing device; and one or more connectors, the one or more connectors to connect the one or more handles to an end of an exercise band or an end of a barbell.

### RELATED APPLICATIONS

This application claims priority to and is related to application Ser. No. 62/814,714, filed Mar. 6, 2019, entitled “Intelligent Exercise or Therapy Apparatus,” the disclosure of which is hereby incorporated by reference.

### BACKGROUND

In the physical therapy and personal training industry, one of the most prominent issues is patient or athlete compliance with exercise or therapy regiments. Physical therapists and trainers cannot track or monitor their clients when they leave the clinic and need to do their exercises. Furthermore, if clients need to travel consistently, it is difficult to maintain a consistent monitoring system to perform exercises.

### BRIEF DESCRIPTION OF THE DRAWINGS

FIG. 1A illustrates a block diagram of an exercise therapy apparatus according to some embodiments;

FIG. 1B illustrates a block drawing of an exercise and therapy apparatus including other components according to some embodiments;

FIG. 1C illustrates an embodiment of the exercise and therapy apparatus according to embodiments;

FIG. 1D illustrates an exercise and therapy apparatus according to some embodiments;

FIG. 1E illustrates a rendering of the exercise and therapy apparatus of FIG. 1C;

FIG. 2 illustrates a block diagram of a therapy and exercise system according to some embodiments;

FIG. 3 illustrates a data flow diagram of how the exercise and therapy measurements, data may be transferred, analyzed, manipulated, and/or stored in memory devices of different computing devices according to some embodiments;

FIG. 4A below illustrates a user performing an exercise according to embodiments utilizing the therapy and exercise apparatus or device described herein;

FIG. 5A illustrates a user performing an exercise, e.g., an elastic band squat utilizing an exercise and therapy device according to embodiments;

FIG. 6A below illustrates a user performing an exercise, e.g., an Elastic Band Low Row exercise, utilizing an exercise and therapy device according to some embodiments;

FIG. 7A below illustrates a user performing an Elastic Band Bicep Curl, utilizing the exercise and therapy apparatus according to some embodiments;

FIG. 8 illustrates a flowchart of a user interacting with an exercise and therapy device along with an exercise and therapy software application according to some embodiments;

FIG. 9 illustrates location of components of an intelligent barbell according to some embodiments;

FIG. 10A illustrates a software system application according to some embodiments;

FIG. 10B illustrates a flowchart of a new exercise mode for the intelligent exercise or therapy software application according to some embodiments;

FIG. 10C illustrates a menu after the user or operator has selected the exercise device (e.g., in this case an intelligent barbell identified as either Smartbell or Joe's Smartbell);

FIG. 10D illustrates an intelligent exercise software application selection of parameters according to some embodiments;

FIG. 10E illustrates an intelligent exercise software application allowing a user to select display of an x-direction or x-angle measurement, a y-angle measurement and/or a user power metric or parameter;

FIG. 10F illustrates a screen requesting that the user or operator select whether or not to process the captured metrics and/or parameters for the selected exercise;

FIG. 10G illustrates an x-angular displacement of an intelligent barbell during the selected exercise according to embodiments;

FIG. 11A illustrates a flowchart of an intelligent exercise software application live mode according to some embodiments;

FIG. 11B illustrates a menu generated by the intelligent exercise software application to allow a user or operator to select an exercise device according to some embodiments;

FIG. 11C illustrates a menu including a live mode, a HEP mode, and/or a calibrate or program mode;

FIG. 11D illustrates the display of a user's x-angle displacement over a period of time utilizing the intelligent barbell for the selected exercise according to some embodiments;

FIG. 11E illustrates the display of a user's y-angle displacement over a period of time utilizing the intelligent barbell for the selected exercise according to some embodiments;

FIG. 12A illustrates a flowchart of an intelligent exercise software application exercise or calibrate mode according to some embodiments;

FIG. 12B illustrates a list of previously created exercise routines for the intelligent exercise software application according to some embodiments;

FIG. 12C illustrates a menu to allow setting of a precision parameters according to some embodiments;

FIG. 13A illustrates an intelligent exercise band according to some embodiments;

FIG. 13B illustrates a handle of an intelligent exercise band according to some embodiments

FIG. 14A illustrates a physical therapist client input screen according to some embodiments;

FIG. 14B illustrates an overview menu of a physical therapy software application according to some embodiments;

FIG. 14C illustrates an exercise description screen or menu according to some embodiments

FIG. 14D illustrates an exercise performance screen or menu according to some embodiments;

FIG. 14E illustrates an exercise summary comparison submenu according to some embodiments;

FIG. 14F illustrates a calculated exercise metrics submenu according to some embodiments; and

FIG. 14G illustrates a video upload submenu according to some embodiments;

### SUMMARY

The subject matter disclosed herein allows for more effective compliance between physical therapists (and/or personal trainers) and their clients. Additionally, the subject matter disclosed herein provides the user, physical therapist, and trainer with biometric information previously unavailable in the market that allow them to effectively debug biomechanical imbalances in performing exercises and correct improper form.

The claimed subject matter is an exercise system and sensor integrated device that can collapse and expand to various forms and shapes. The physical design is unique to the market. In some embodiments, the exercise and therapy device allows a barbell form to change length, and split into handles that can be used on any cable machine. In some embodiments, the exercise and therapy device further comprises resistance bands. In addition, in some embodiments, the exercise and therapy device may be utilized as the mid-section of a dumbbell system. The modularity of the exercise and therapy device allows its use and application in many rehabilitation, mobility, strength, and/or fitness exercises. An additional novel and/or unique aspect of the exercise and therapy device is its ability to automatically measure the weight lifted by the user, whereas prior devices require users to manually enter the weight interacted with into software applications for analysis and/or tracking.

In embodiments, the exercise and therapy device may have one or more sensors that capture measurements and parameters while a user performs or executes an exercise. In some embodiments, the exercise and therapy device may comprise one or more processors or microcontrollers, one or more memory devices, and/or computer-readable instructions stored in the one or more memory devices that are executable by the one or more processors to perform certain tasks. In some embodiments, the one or more processors or microcontrollers may be coupled to the one or more sensors and may read raw measurements or parameters from the one or more sensors integrated within or as part of the exercise and therapy device. In some embodiments, the exercise and therapy device may comprise one or more wireless communication transceivers (e.g., a personal area network transceiver or a wireless LAN transceiver). In embodiments, the one or more processors may be coupled to the one or more wireless communication transceivers. In some embodiments, computer-readable instructions executable by the one or more processors or microcontrollers may communicate the received raw measurements or parameters to an external computing device for post-processing and analyzation. In some embodiments, the raw measurements or parameters may be multi-axis angular measurements or parameters and location measurements received from one or more gyroscopes, accelerometers and/or directional sensors; grip strength and/or weight lifted measurements or parameters obtained by pressure sensors and/or strain gauges; and/or velocity and/or acceleration measurements or parameters from accelerometers. In some embodiments, the raw measurements and/or parameters from the sensors may be processed and biometrics, which have been unavailable in the exercise and therapy market, may be calculated, analyzed and/or presented to the user, physical therapist, or trainer. In some embodiments, the raw measurements and/or parameters may be stored in an external computing device and/or cloud database computing device.

In some embodiments, computer-readable instructions executable by one or more processors on the external computing device may calculate biometrics parameters and/or measurements. In some embodiments, for example, the biometrics parameters and measurements derived from the sensors may be in the form of three-axis angle deviation, acceleration, velocity, and/or position tracking measurements and/or parameters. In some embodiments, the three-axis angle deviation, acceleration, velocity and/or position tracking measurements and/or parameters may be obtained and/or calculated real-time and may be stored in one or more memory devices of the external computing device. Additionally, in some embodiments, biometrics measurements and/or parameters derived from pressure sensors and/or strain gauges may be in the form of grip strength, weight lifted and/or power output measurements obtained or calculated in real-time and may be stored in the one or more memory devices of the external computing device.

In embodiments, computer-readable instructions executable by one or more processors of the external computing device or a cloud-based server computing device may analyze, calculate and/or present (on a display or monitor of the external computing device) the calculated biometric measurements and/or parameters to a user, physical therapist, or trainer in real-time in the form of one, two, or three-dimensional graphs and performance scores. In embodiments, the biometrics measurements and/or parameters may be plotted alongside and compared with correct form profiles and ranges to inform the user, physical therapist, or trainer on the precision and accuracy of the execution of the exercise. In some embodiments, computer-readable instructions executable by one or more processors on the external computing device may generate a verbal recommendation on how to correct the execution of the exercise which may be presented audibly to the user, physical therapist, or trainer utilizing speakers on the external computing device. In embodiments, computer-readable instructions executable by one or more processors on the external computing device may communicate commands, instructions or messages to the exercise and therapy device to guide the user in performing the exercise correction. In embodiments, the exercise and therapy device may comprise a vibration motor which may receive instructions or signals to provide local haptic feedback to the user. In embodiments, the exercise and therapy device may comprise one or more lighting elements or assemblies and the one or more lighting elements or assemblies may receive instructions or signals to generate visual feedback (such as illuminating LEDs on certain parts of the exercise and therapy device) to identify when deviations from correct movements are detected by the exercise and therapy system. In some embodiments, there may be different levels of feedback that may be provided to a user. First example, depending on how far off the user or patient is from the correct readings or measurements, different levels of signals may be communicated. For example, if the measurements or parameters received from the exercise and therapy device are within 0 to 3 to 5% of what is expected, the exercise and therapy device (or the exercise and therapy device software) may not generate error messages or warning messages through the one or more speakers, LEDs or vibration motors. In some embodiments, if the measurements or parameters are within 3 to 5% up to 15 percent, the exercise and therapy device (or the exercise and therapy device software) may generate a low intensity audible warning, a low intensity visual warning through the LEDs and/or a low intensity vibration through the vibration motor. In some embodiments, if the measurements or parameters are greater than 15% from the expected measurements or parameters, then the exercise and therapy device (or the exercise and therapy device software) may generate a high intensity audible warning, a high intensity visual warning through the LEDs and/or a high intensity vibration through the vibration motor in order to stop the user or operator to continue from performing the exercise incorrectly. The exercise and therapy system may be the combination of the software running on the external computing device and the exercise and therapy apparatus or device.

### DETAILED DESCRIPTION

The foregoing, and other features and advantages of the invention and claimed subject matter, will be apparent from the following, more particular description of the preferred embodiments of the invention and claimed subject matter, the accompanying drawings, and the claims.

In the following detailed description, numerous specific details are set forth to provide a thorough understanding of claimed subject matter. For purposes of explanation, specific numbers, systems and/or configurations are set forth, for example. However, it should be apparent to one skilled in the relevant art having benefit of this disclosure that claimed subject matter may be practiced without specific details. In other instances, well-known features may be omitted and/or simplified so as not to obscure claimed subject matter. While certain features have been illustrated and/or described herein, many modifications, substitutions, changes and/or equivalents may occur to those skilled in the art. It is, therefore, to be understood that appended claims are intended to cover any and all modifications and/or changes as fall within claimed subject matter.

References throughout this specification to one implementation, an implementation, one embodiment, embodiments, an embodiment and/or the like means that a particular feature, structure, and/or characteristic described in connection with a particular implementation and/or embodiment is included in at least one implementation and/or embodiment of claimed subject matter. Thus, appearances of such phrases, for example, in various places throughout this specification are not necessarily intended to refer to the same implementation or to any one particular implementation described. Furthermore, it is to be understood that particular features, structures, and/or characteristics described are capable of being combined in various ways in one or more implementations and, therefore, are within intended claim scope, for example. In general, of course, these and other issues vary with context. Therefore, particular context of description and/or usage provides helpful guidance regarding inferences to be drawn.

Likewise, in this context, the terms “coupled”, “connected,” and/or similar terms are used generically. It should be understood that these terms are not intended as synonyms. Rather, “connected” is used generically to indicate that two or more components, for example, are in direct physical, including electrical, contact; while, “coupled” is used generically to mean that two or more components are potentially in direct physical, including electrical, contact; however, “coupled” is also used generically to also mean that two or more components are not necessarily in direct contact, but nonetheless are able to co-operate and/or interact. The term “coupled” is also understood generically to mean indirectly connected, for example, in an appropriate context.

The terms, “and”, “or”, “and/or” and/or similar terms, as used herein, include a variety of meanings that also are expected to depend at least in part upon the particular context in which such terms are used. Typically, “or” if used to associate a list, such as A, B or C, is intended to mean A, B, and C, here used in the inclusive sense, as well as A, B or C, here used in the exclusive sense. In addition, the term “one or more” and/or similar terms is used to describe any feature, structure, and/or characteristic in the singular and/or is also used to describe a plurality and/or some other combination of features, structures and/or characteristics. Likewise, the term “based on,” “based, at least in part on,” and/or similar terms (e.g., based at least in part on) are understood as not necessarily intending to convey an exclusive set of factors, but to allow for existence of additional factors not necessarily expressly described. Of course, for all of the foregoing, particular context of description and/or usage provides helpful guidance regarding inferences to be drawn. It should be noted that the following description merely provides one or more illustrative examples and claimed subject matter is not limited to these one or more illustrative examples; however, again, particular context of description and/or usage provides helpful guidance regarding inferences to be drawn.

Mobile computing device, smartphones, tablets, network devices, and/or mobile communications device may be used interchangeably herein to refer to a computing device that is portable that includes computing (e.g., processing capability) and/or communications capability (e.g., both cellular and data communications). In some embodiments, other computing devices may be utilized in the system such as laptop computer devices, desktop computing devices, and/or other non-mobile computing devices that may run the software. In some embodiments, microcontrollers, microprocessors, controllers, and/or processors may be utilized interchangeably herein.

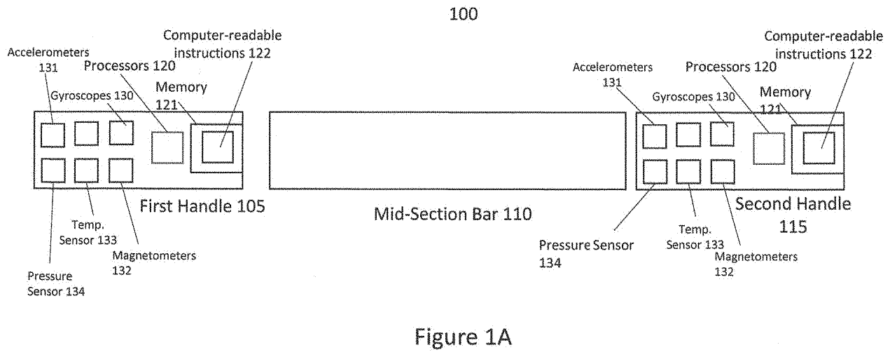

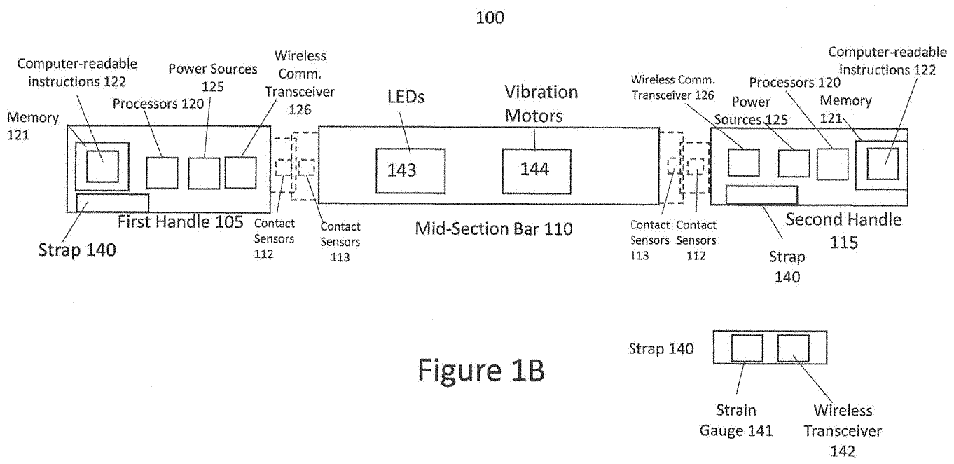

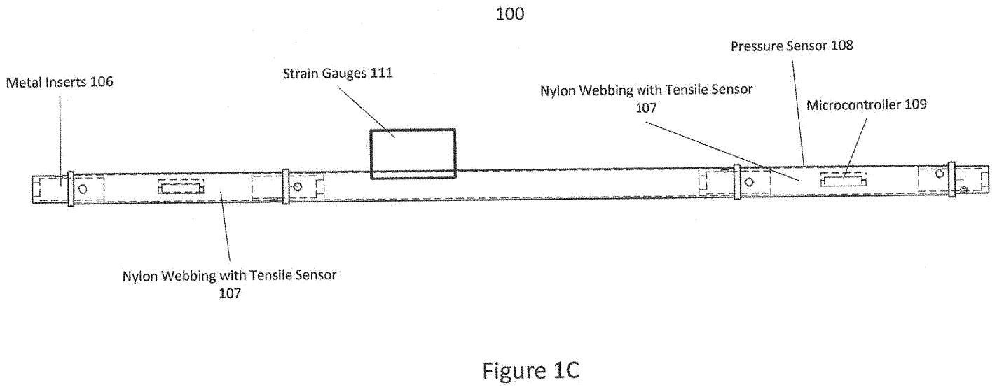

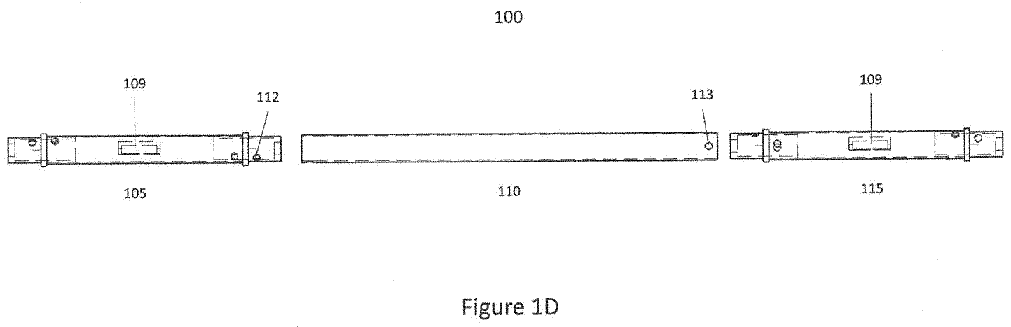

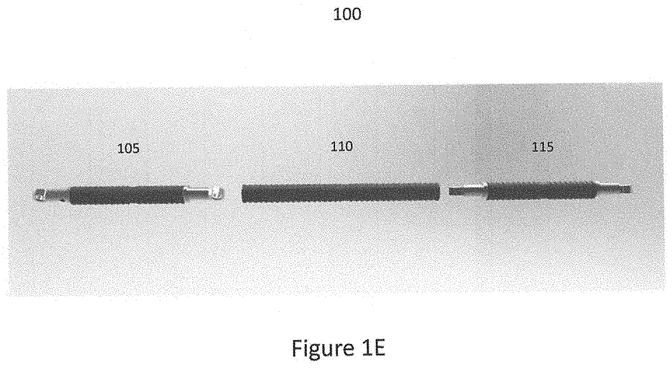

FIG. 1A illustrates a block diagram of an exercise and therapy apparatus according to some embodiments. FIG. 1E illustrates a physical rendering of an exercise and therapy apparatus according to some embodiments. FIG. 1B illustrates a block drawing of an exercise and therapy apparatus including other components according to some embodiments. In some embodiments, the exercise and therapy apparatus 100 comprises a first handle 105, a mid-section 110 and a second handle 115. In some embodiments, the first handle 105 may be interlocked with a first end of the midsection 110 via a pin-sprung locking mechanism or assembly and the second handle 115 may be interlocked with a second end (e.g., an opposite end to the first end) of the midsection 110. In some embodiments, one or more contact sensors 112 may be located in a first handle 105 and/or a second handle 115 and one or more contact sensors 113 may be located on each end of a mid-section bar 110. In embodiments, when the first handle 105 is connected or attached to the mid-section bar 110 (or when the second handle 115 is connected or attached to the mid-section bar 110), the contact sensor 112 in the handles makes contact or a connection with the contact sensor 113 in the mid-section bar 110 and generates a signal or message that the mid-section bar 110 and the first handle 105 (and/or the second handle 115) are in contact with and/or attached to the mid-section bar 110. In some embodiments, the contact sensors 112 and/or 113 may communicate message and/signals to one or more microcontrollers located in the first handle 105 and/or the second handle 110.

In embodiments, the first handle 105 and/or the second handle 115 may have a length ranging from 1 inch to 12 inches. In embodiments, the first handle 105 and the second handle 115 may have a diameter ranging from 0.10 inches to 2 inches. In some embodiments, the first handle 105 and the second handle 115 may be made of a metal material (e.g., a lightweight metal material). In some embodiments, the first handle 105 and the second handle 115 may be made of a composite material and/or a plastic material. In some embodiments, the first handle 105 and the second handle 115 may have a hollow interior. In some embodiments, the hollow interior may allow many electrical components (e.g., microcontroller, power sources, and/or sensors) to be located and/or housed in the interior of the first handle 105 and/or the second handle 115.

In some embodiments, one or more nylon straps 140 may be located or stored in the hollow interior of the first handle 105 and/or the second handle 115. In some embodiments, the one or more straps 140 may be attached to an exterior surface of the first handle 105 and/or the second handle 115 in order to perform physical exercise and/or therapy exercises. In some embodiments, the one or more nylon straps 140 may be connected via fasteners to hooks or other similar connectors on or attached to exterior surfaces of the first handle 105 and/or second handle 115. In some embodiments, the one or more straps 140 may be flexible and/or bendable to allow for placement and/or storage in the first handle 105 and/or the second handle 115. In some embodiments, the one or more straps 140 may comprise one or more strain gauges 141 to measurement force or strain being exerted when utilizing the one or more straps 140. In some embodiments, the one or more strain gauges 141 may include one or more communication transceivers 142 to communicate any measurements, indicators or parameters to the one or more microcontrollers in the first handle 105 and/or the second handle 115. In some embodiments, the one or more strain gauges may be mechanically and/or electrically connected to the one or more microcontrollers in the first handle 105 and/or the second handle 115.

In some embodiments, the mid-section bar 110 may be made of a metal material. In some embodiments, the mid-section bar 110 may be made of a plastic and/or a composite material. In some embodiments, the mid-section bar may be solid and not have a hollow interior. In some embodiments, the mid-section bar 110 may have a hollow interior in which to store other exercise or therapy devices and/or one or more electrical components. In some embodiments, the mid-section bar 110 may have different weights so that different mid-section bars 110 (and thus different weighted bars) may be attached to the first handle 105 and the second handle 115 to perform different exercises. In some embodiments, the mid-section bar 110 may have a length ranging from 5 inches to 60 inches. In some embodiments, the mid-section bar 110 may have a diameter of 0.1 inches to 2.0 inches.

In some embodiments, the midsection 110 (or mid-section bar) may be different configurations (e.g., different lengths and/or different widths). In some embodiments, the first handle 105 and the second handle 115 may be separable from the midsection 110 and may be used independently and thus for different patient exercises. In some embodiments, the first handle 105 and the second handle 115 may be utilized independently and/or in combination with the mid-section bar 110, along with cables, and/or resistance bands, and other exercise devices or systems. In some embodiments, the first handle 105 and the second handle 115 may have fabric grips. In some embodiments, the fabric may be a nylon material, a rubber material and/or other similar materials.

In some embodiments, pressure sensitive material (or strain gauges 111) may be embedded inside the fabric or material grips on the first handle 105 and the second handle 115. In some embodiments, the pressure sensitive material (or strain gauges 111) may measure force being exerted by the patient on the first handle 105 and/or the second handle 115. In some embodiments, the pressure sensitive material (or strain gauge 111) may generate a signal and/or message based at least one part on the measured force which may be communicated to the one or more microcontrollers in the first handle 105 and/or the second handle 115.

In some embodiments, the first handle 105 and the second handle 115 may comprise one or more gyroscopes 130 (e.g., a three-axis gyroscope), one or more accelerometers 131 (e.g., a three-axis accelerometer), one or more magnetometers 132 (e.g., a three-axis magnetometer), one or more pressure sensors 134, and/or temperature sensors 133. In some embodiments, the first handle 105 and the second handle 115 may further comprise one or more microcontrollers or processors 120, one or more memory devices 121, one or more wireless communication transceivers 126 and/or one or more batteries and/or one or more rechargeable batteries 125. In some embodiments, the one or more gyroscopes 130, one or more accelerometers 131, one or more magnetometers 132, one or more pressure sensors 134, and/or temperature sensors 133 may be located or positioned in an interior of the first handle 105 and/or the second handle 115. In some embodiments, the one or more gyroscopes 130, one or more accelerometers 131, one or more magnetometers 132, one or more pressure sensors 134, and/or temperature sensors 133 may be attached to a surface of the first handle 105 and/or the second handle 115. In some embodiments, some of the sensors may be located inside the first handle 105 and/or the second handle 115 and some of the sensors may be attached to an outside surface of the first handle 105 and/or the second handle 115. In some embodiments, computer-readable instructions executable by the one or more processors 120 may read or receive measurements, data and/or parameters from the one or more three-axis accelerometers 131, one or more three-axis gyroscopes 130, one or more three-axis magnetometers 132, one or more pressure sensors 134, and one or more temperature sensors 133 integrated inside each handle (e.g., 105 and 115) (or placed outside each handle) as well as from one or more pressure strain gauges 108 integrated within the rubber grips 111 of the first handle 105 and the second handle 115.

In some embodiments, the one or more microcontrollers or processors 120, the one or more memory devices 121, the one or more wireless communication transceivers 126 and/or the one or more power sources 125 (e.g., rechargeable batteries) may be located in an interior portion of the first handle 105 and/or the second handle 115 (e.g., in the hollow portion). In embodiments, these devices may be integrated onto a single chip or board. In some embodiments, the one or more microcontrollers 120, the one or more memory devices 121, the one or more wireless communication transceivers 126 and/or the one or more power sources 125 may be attached to an outer surface of the first handle 105 and/or the second handle 115. In some embodiments, computer-readable instructions 122 may be stored in one or more memory devices 121 and/or may be executable by the one or more processors and/or microcontrollers 120 to perform operations and/or interact with the various sensors and/or one or more wireless communication transceivers 126. In some embodiments, the computer-readable instructions 122 executable by the one or more microcontrollers or processors 120 may activate and/or deactivate the sensors, may receive measurements, parameters and/or data from the sensors, may store the received sensor measurements, parameters and/or data in one or memory devices 121 and/or may communicate the received sensor measurements, parameters and/or data via the one or more wireless communication transceivers 126 to one or more external computing devices as will be described later.

FIG. 1C illustrates an embodiment of the exercise and therapy apparatus according to embodiments. In some embodiments, the first handle 105 and the second handle 115 may comprise one or more metal inserts 106, one or more fabric webbing 107 including a tensile sensor (or pressure sensor) 108 and a microcontroller device 109). In embodiments, the microcontroller device 109 may comprise the one or more microcontrollers or processors, one or more memory devices and/or computer-readable instructions described above. Because FIG. 1E is a rendering, there are no pictures or illustrations of the sensors and/or microcontrollers that are resident and/or positioned in an interior of the exercise and/or therapy apparatus. FIG. 1D illustrates the exercise and therapy apparatus of FIG. 1C. FIG. 1D illustrates a possible position of contact sensors 112 in the first handle 105 as well as the possible position of the contact sensors 113 in the mid-section bar 110.

In some embodiments, the first handle 105, the mid-section bar 110, and the second handle 115 may comprise one or more lighting elements 143 and one or more vibration motors 144. In embodiments, computer-readable instructions executable by one or more processors of an external computing device (or database servers and/or cloud-based servers) may detect and/or identify that out-of-tolerance measurements and/or incorrect measurements, parameters or data may have been received from the one or more sensors on the exercise and therapy apparatus. In some embodiment, computer-readable instructions executable by one or more processors of the external computing device (or database servers and/or cloud-based servers) may generate instructions, messages and/or signals and communicate the instructions, commands, messages and/or signals to the exercise and therapy apparatus 100 to provide feedback to the user regarding incorrect operation or potential correction instructions of the exercise and therapy apparatus 100. In embodiments, the exercise and therapy device 100 may comprise a vibration motor 144 which may receive instructions or signals to provide local haptic feedback to the user. In embodiments, the exercise and therapy device may comprise one or more lighting elements or assemblies 143 and the one or more lighting elements or assemblies 143 may receive instructions or signals to generate visual feedback (such as illuminating LEDs on certain parts of the exercise and therapy device) to identify when deviations from correct movements are detected by the exercise and therapy system. In some embodiments, there may be different levels of feedback that may be provided to a user. For example, depending on how far off the user or patient is from the correct readings or measurements, different levels of signals may be communicated. For example, if the measurements or parameters received from the exercise and therapy device are within 0 to 3 to 5% of what is expected, the exercise and therapy device (or the exercise and therapy device software) may not generate error messages or warning messages through the one or more speakers, LEDs or vibration motors. In these cases, the exercise and therapy software is determining that these measurements are not out-of-tolerance. In some embodiments, if the measurements or parameters are within 3 to 5% up to 15 percent, the exercise and therapy device (or the exercise and therapy device software) may generate a low intensity audible warning, a low intensity visual warning through the LEDs and/or a low intensity vibration through the vibration motor. In some embodiments, if the measurements or parameters are greater than 15% from the expected measurements or parameters, then the exercise and therapy device (or the exercise and therapy device software) may generate a high intensity audible warning, a high intensity visual warning through the LEDs and/or a high intensity vibration through the vibration motor in order to stop the user or operator to continue from performing the exercise incorrectly. In some embodiments, these ranges may be adjusted by the user or by the operator of the exercise and therapy software and system in that some measurements may need to be performed with much tighter tolerance (e.g., if measurements are not within 0.5%, low intensity warnings may be generated). The above-numerical ranges are just representative examples.

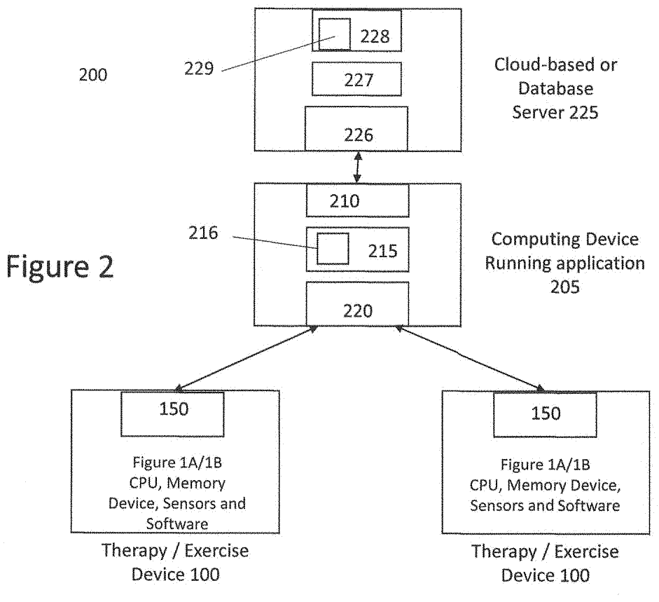

FIG. 2 illustrates a block diagram of a therapy and exercise system according to some embodiments. FIG. 2 illustrates how the exercise and therapy apparatus or device 100, a user computing device 205, and/or a database may be used. In some embodiments, each exercise and therapy apparatus or device 100 may consist of all the sensors described with respect to FIGS. 1A and 1B, as well as one or more wireless transceivers 150 (e.g., one or more wireless transmitters or one or more wireless receivers), and one or more memory devices 121. In some embodiments, the measurements, parameters and/or data captured by the sensors of the exercise and therapy apparatus or device 100 may be communicated by the one or more wireless transceivers 150 to user computing device 205. In some embodiments, the user computing device 205 may be a smartphone, tablet, wearable computing device, or any external computing device. In some embodiments, the user computing device 205 may comprise one or more processors 210, one or more memory devices 215, communication circuitry or transceivers 220, and/or computer-readable instructions 216 executable by one or more processors. In some embodiments, the computer-readable instructions 216 executable by one or more processors may be exercise and therapy application software and/or user interface software. In embodiments, the exercise and therapy measurements, parameters, and data received from the one or more sensors or the one or more exercise and therapy apparatus may be stored in the one or more memory devices 215 of the user computing device 205. As shown in FIG. 2, the user computing device may forward and/or communicate the measurements, parameters and/or data the user computing device collects and/or captures to a server computing device or database 225 via communication circuitry 226 (e.g., wireless or wired communication transceivers). In some embodiments, computer-readable instructions 229 stored in the one or more memory devices 228 may be executable by one or more processors 227 may interface and/or interact with application software of the user computing device. In some embodiments, the user computing device 205 may also be able to communicate and/or interface with a multiplicity or plurality of (N-devices) of exercise and therapy devices 100 which help track exercise performance. FIG. 2 illustrates communication with two or exercise and therapy devices 100.

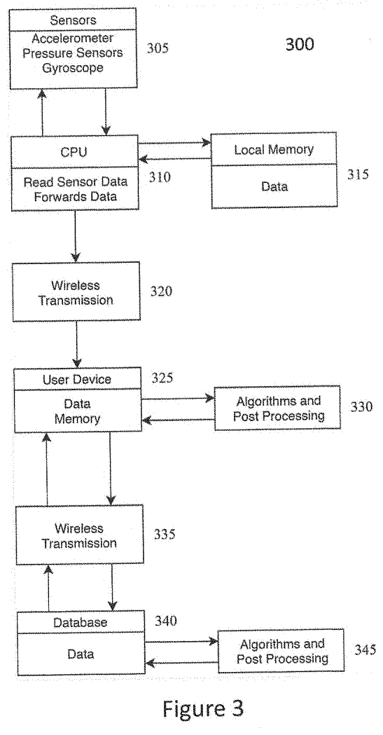

FIG. 3 illustrates a data flow diagram of how the exercise and therapy measurements, data may be transferred, analyzed, manipulated, and/or stored in memory devices of different computing devices. For example, in step 305, the above-mentioned sensors (including but not limited to one or more gyroscopes, one or more pressure sensors, and/or one or more accelerometers) may capture measurements, parameters and/or data while a subject is performing an exercise. In embodiments, in step 310, computer-readable instructions executable by one or more microcontrollers or processors (or CPUs) may read the sensor measurements, parameters and/or data and in step 315, may store the sensor measurements, parameters and/or data in one or more local memory devices of the exercise and therapy apparatus or device. In some embodiments, in step 320, computer-readable instructions executable by the one or more processors may wirelessly communicate the sensor measurements, parameters and/or data to other computing devices (including but not limited to user computing devices, such as a laptop, smartphone, tablet and/or desktop computing devices).

In some embodiments, in step 325, computer-readable instructions executable by one or more processors of the user computing device may receive the sensor measurements, parameters and/or data and may store the received sensor measurements in one or more memory devices of the user computing device. In embodiments, in step 330, computer-readable instructions executable by the one or more processors on the user computing device may perform post-processing on the received sensor measurements, parameters and/or data to create analyzed exercise and/or therapy data and/or information. In some embodiments, in step 335, computer-readable instructions executable by the one or more processors of the user computing device may communicate, via wireless transmission, the analyzed exercise and/or therapy data and/or information (as well as the sensor measurements, parameters and/or data) to remote database servers (and/or cloud-based servers). In embodiments, in step 340, computer-readable instructions executable by one or more processors of the database server may store the analyzed exercise and/or therapy data and/or information (as well as the sensor measurements, parameters and/or data) in one or more memory devices and/or relational databases). In embodiments, in step 345, computer-readable instructions executable by one or more processors of the database servers (and/or cloud-based servers) may perform additional processing and/or analyzation of the analyzed exercise and/or therapy data and/or information received from the user computing device (as well as the sensor measurements, parameters and/or data) in order to generate aggregated analyzed exercise and/or therapy data and/or information.

FIG. 4A below illustrates a user performing an exercise according to embodiments utilizing the therapy and exercise apparatus or device described herein. In embodiments, which may be referred to as use case example 1, a subject or individual may be performing an Elastic Band External Rotation with Abduction exercise. In some embodiments, a user may utilize one of the handles 105 or 115 of the exercise and/or therapy apparatus device along with a resistance band to perform the exercise. In some embodiments, the user or subject may fix or connect one end of a resistance band a distance away such that the appropriate tension in the exercise band is reached. In some embodiments, one end of the resistance band may be connected to a permanent or stable support (such as the railing support illustrated in FIG. 4A) and the other end of the resistance band may be connected or coupled to one of the handles of the exercise and/or therapy apparatus. In some embodiments, in this exercise, the user may attempt to hold their forearm at a ninety-degree angle to their bicep and an in a plane parallel to the band. In some embodiments, in this exercise, the user may increase the tension in the exercise band as they rotate their forearm until it is nearly perpendicular to the ground surface (as is shown in FIG. 4A). In some embodiments, in this exercise, the user may repeat this motion to improve mobility and strength over a specified time period.

The measurement, parameter and data acquisition and feedback from the exercise and therapy device received during the exercise is described here. In embodiments, a linking of the exercise and therapy apparatus and the user computing device may be established, either automatically and/or through user selection. In embodiments, once the exercise and therapy device is connected to external device, the user, in the exercise and therapy software application, may select an exercise (e.g., the exercise described above) and trigger the start of an exercise or therapy session where measurements, parameters and data may be recorded. Once an exercise or therapy session has been initiated or triggered, the sensors (described above) may be initialized and/or activated to capture the measurements, parameters and/or data. During the exercise, the measurements, parameters and/or data may be captured by the sensors, recorded in one of the memory devices (of the exercise and/or therapy device, the user computing device, and/or the database server), and/or analyzed by the executable computer-readable instructions of either the user computing device and/or the database server. In some embodiments, the results of the analyzation may be displayed or plotted in a user software application running or executing on the user computing device and will be recorded and plotted in the user application on an external device.

1^{st} Use Case—Described immediately below are the measurements, parameter and/or data being captured by the sensors of the exercise and therapy device for the above-identified exercise. In some embodiments, measurements, parameters, or data from strain gauges or pressure sensors embedded in the cables or straps of the exercise and therapy device may indicate the tension in cables or straps during an exercise. In some embodiments, the tension measurements, parameters, data and/or readings may be recorded or stored in the one or memory devices described above. In embodiments, after the tension measurements, parameters, data and/or readings have been transmitted at least to a user's computing device, computer-readable instructions executable the one or more processors of the computing device may track and/or plot the tension measurements, parameters, data and/or readings the users received tension measurements, parameters, data and/or readings over time as well as the strength improvement over time.

As an additional example, three-axis angular position measurements, parameters and/or data may be captured from the one or more gyroscopes, accelerometers, and magnetometers. In embodiments, the three-axis angular position measurements, parameters and/or data may be stored in the one or more memory devices of the exercise and therapy device, the user computing device (after transmission) and/or the database server (after transmission). In embodiments, computer-readable instructions executable by one or more processors on the user computing device (and/or the database server) may be plotted and/or compared against an acceptable range of angular parameters, measurements and/or data range for this exercise. In embodiments, computer-readable software executable by one or more processors of the exercise and therapy apparatus and/or the user computing device may sample, analyze and/or review the angular measurements of the handle being used to ensure and/or verify that the handle is remaining parallel to the ground and perpendicular to the resistance band as required by this exercise.

In embodiments, a pressure sensitive material (or pressure sensor) and/or a strain gauge may capture a user's grip force measurements, parameters and/or data throughout the exercise. In embodiments, the user's grip force measurements, parameters and/or data may be stored in the memory devices of the exercise and therapy apparatus, the user's computing device and/or the database server. In addition, after the user's grip force measurements, parameters, and/or data have been transmitted to the user's computing device and/or database server, computer-readable instructions executable by the one or more processors on these devices may track or plot the received user's grip force measurements, parameters and/or data against acceptable ranges to verify and/or ensure a stable grip is being maintained for the exercise. In embodiments, relative improvements in grip strength and comparisons to the opposing arm can be used to better guide the recovery of an injury, biomechanical imbalance, or show improvements in strength.

In embodiments, sensor measurements, parameters and/or data form accelerometers, gyroscopes, magnetometers and/or pressure sensitive materials may be utilized to calculate a power output after these measurements, parameters and/or data are received from the exercise and therapy apparatus. In embodiments, computer-readable instructions executable by one or more processors on a user's computing device and/or database server may calculate the power output based at least in part on these sensor measurements, parameters and/or data form accelerometers, gyroscopes, magnetometers and/or pressure sensitive materials/strain gauges. In embodiments, a sample or illustrative calculation of power output is shown below where Acceleration. x represents the acceleration in the x direction.

Power=(Tension)(Acceleration. x²+Acceleration. y²+Acceleration. z²)^{1/2}(Sample time)

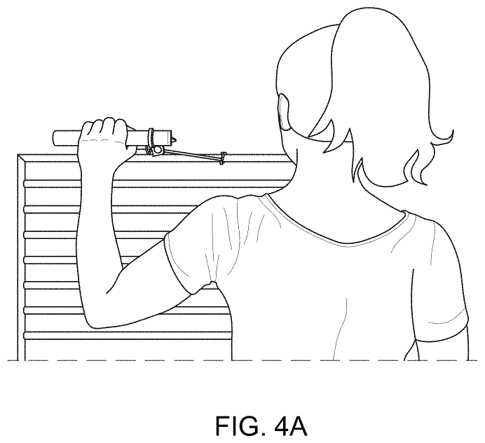

2^{nd} Use Case—FIG. 5A illustrates a user performing an exercise, e.g., an elastic band squat utilizing an exercise and therapy device according to embodiments. In some embodiments, the exercise and therapy device will be utilized as a complete barbell (e.g., the left and right handles 105 and 115 and the mid-section). In embodiments, a resistance band may connect or attach outside ends of the right handle and the left handle. In some embodiments, in this exercise, the user may place the exercise and therapy device (as a barbell) on his or her shoulders as well as stepping on the exercise band which may create tension between the exercise and therapy device bar bell and the ground surface. In embodiments, as is shown in FIG. 5A, a user may then perform the elastic band squat which is designed to improve a user's mobility as well as leg strength.

In some embodiments, the exercise and therapy device barbell may be gathering measurements, parameters and/or data during the performance of the elastic band squat exercise. In some embodiments, once the user and therapy device barbell is coupled, connected and/or communicating with the user computing device (or other computing device), a user may select (from the exercise and therapy software application), the elastic band squat exercise and initiate and/or trigger the start of an exercise session. In some embodiments, once an exercise session has been initiated and/or triggered, the sensors in the exercise and therapy device barbell may be initialized and/or activated. In some embodiments, during a user's performance of the elastic band squat exercise, several readings, parameters, measurements and/or data may be recorded and/or stored in memory devices of the exercise and therapy device, the user computing device and/or database server or cloud-based servers. In embodiments, computer-readable instructions executable by the one or more processors of the user computing device (e.g., the software application) and/or database servers may plot or display the received measurements, parameters and/or data received from the one or more sensors in the exercise and therapy apparatus.

In some embodiments, when the left or right handles 105 and 115 (or bars) attach to the mid-section 110, the sensor readings, measurements, and/or parameters may be recorded and transmitted by one/or both of the microcontrollers or processors in the left and/or right handle. In some embodiments, strain gauges along the bar (e.g., the outer surface of the exercise and therapy device) may measure the tension and/or load created by the resistance band. In some embodiments, for example, as the exercise and therapy device bar bends slightly due to the weight of the user, the strain gauge measurements, parameters and/or data may relate that bending to a weight correlation (or load correlation). In some embodiments, the strain gauge located at the center of the mid-section 110 will be connected to the microcontroller or processor in one or both of the handles by an isolated metal contact on the ends of 105, 110, 115.

In some embodiments, the one or more gyroscopes, accelerometers, and magnetometers may generate three-axis angular position measurements, parameters and/or data. In some embodiments, software executable by one or more processors or controllers of the exercise and therapy device may communicate the three-axis angular position measurements, parameters and/or data to the user computing device. In some embodiments, exercise and therapy software executing on the user computing device may plot and/or display the angular position measurements, parameters and/or data recorded from each axis against an acceptable range for this resistance band squat exercise. In some embodiments, software executing on either or both of the exercise and therapy device and/or the user computing device may sample and/or verify that the angular position measurements, parameters and/or data associated with the handle of the exercise and therapy device to ensure that the handle of the exercise and therapy device is remaining parallel to the ground and perpendicular to the resistance band as required by the resistance band squat exercise.

In some embodiments, one or more pressure sensors (e.g., pressure sensitive material) or a strain gauge may capture a user's grip force measurements, parameters and/or data. In embodiments, the user's grip force measurements, parameters and/or data may track and/or monitor this during the resistance band squat exercise to ensure a stable grip is being utilized throughout the exercise. In some embodiments, software executing on the one or more processors and/or controllers of the exercise and therapy device may communicate the captured grip force measurements, parameters and/or data to the user's computing device via wireless communications in order to analyze and/or track the user's grip strength. In some embodiments, the relative improvements in grip strength and comparisons to the opposing arm can be used to better guide the recovery of an injury, biomechanical imbalance, or show improvements in strength in the arms.

In some embodiments, accelerometer, gyroscope, magnetometer, and pressure sensitive material, and/or strain gauge measurements, parameters and/or data may be utilized to calculate a user's power output. In embodiments, the accelerometers, gyroscopes, magnetometers, pressure sensitive materials, and/or strain gauges may capture the associated measurements, parameters and/or data and software executing on the processors or controllers of the exercise and/or therapy device may communicate the associated measurements, parameters and/or data to the user's computing device. In embodiments, exercise and therapy software executing on the user's computing device may analyze and/or display the associated measurements, parameters and/or data to the user. In some embodiments, the exercise and therapy software may utilize an algorithm similar to the one shown below to calculate a user's power output. A sample calculation of power is shown below where Acceleration.x represents the acceleration in the x direction.

Power=(Tension)(Acceleration.x²+Acceleration.y²+Acceleration.z²)^{1/2}(Sample time)

Use Case 3: Elastic Low Rows—FIG. 6A illustrates a user performing an exercise, e.g., an Elastic Band Low Row exercise, utilizing an exercise and therapy device according to some embodiments. In some embodiments, a user may utilize both of the handles 105 and 115 of the exercise and/or therapy apparatus device along with a resistance band to perform the exercise. In some embodiments, one end of the resistance band may be fixed a distance away from the user such that the appropriate tension is reached as shown in FIG. 6A. In some embodiments, in this exercise, the user will begin each repetition with their arms parallel (forearms and biceps nearly or fully parallel) to the plane of the resistance band. In some embodiments, the user may increase the tension in the band as they rotate their arms to be perpendicular to the ground surface (as shown in FIG. 6A.)

In some embodiments, in the exercise shown in FIG. 6A, the exercise and therapy device may be gathering measurements, parameters, and/or data during the user's performance of the Elastic Low Row. In some embodiments, once the handles 105 and 115 have been coupled, connected, and/or communicating with the user's computing device, the Elastic Low Row exercise may be selected from a list of exercises presented on the exercise and therapy software application installed on the user's computing device. Once the exercise has been triggered and/or initiated on the user's computation device, the sensors in the handles 105 and 115 may be initialized and/or activated. In some embodiments, during a user's performance of the Elastic Low Row, several readings, parameters, measurements and/or data may be recorded and/or stored in memory devices of the exercise and therapy device, the user computing device and/or database server or cloud-based servers. In some embodiments, the received measurements, parameters, and/or data received from the sensors in the exercise and therapy apparatus may be plotted or displayed in the software application by computer-readable instructions executable by the one or more processors on the user computing device.

In some embodiments, tension in the cables of the handles, grip force, and the user's power output may be measured and captured plus communicated to the user computing device and/or database server or cloud-based servers. This is in a similar fashion as described in use case 1, during FIG. 4A. In some embodiments, the one or more gyroscopes, accelerometers, and magnetometers may generate three-axis angular position measurements, parameters and/or data and may communicate the three-axis angular position measurements, parameters and/or data to the user computing device (and/or database server or cloud-based servers) so that an instructor or teacher ensures the first handle 105 and the second handle 115 remain parallel to the ground surface throughout the Elastic Low Row exercise. In some embodiments, the tension and/or three-axis angular position measurements, parameters, and/or data may be recorded, transmitted, processed, and plotted using computer-readable instructions executable by the one or more processors of the user computing device (e.g. software application) and/or the computer-readable instructions executable by the one or more processors of the database server computing device or cloud-based server (e.g., software application) as in stated in prior use case examples.

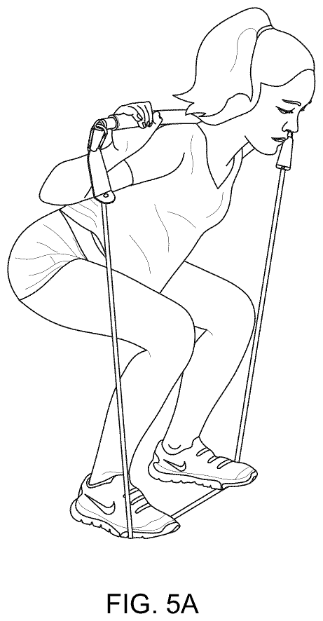

Use case 4: Elastic Band Bicep Curl—FIG. 7A illustrates a user performing an Elastic Band Bicep Curl, utilizing the exercise and therapy apparatus according to some embodiments. In some embodiments, a user may utilize both of the handles 105 and 115 of the exercise and/or therapy apparatus device along with a resistance band to perform the exercise. This exercise is shown in FIG. 7A. In some embodiments, the user will grip both handles 105 and 115, place their arms at their side so that they are perpendicular to the ground surface, step on the resistance band, and curl their forearms upwards, increasing tension in the resistance band, at least until a ninety-degree angle is formed by the forearm and bicep. In some embodiments, the user may maintain a parallel relationship between the handle and ground surface throughout the Elastic Band Bicep Curl exercise.

In some embodiments, in the Bicep Curl exercise shown in FIG. 7A, the exercise and therapy device may be gathering measurements, parameters, and/or data during the user's performance of the Elastic Band Bicep Curl. In some embodiments, the one or more straps 140 that are housed in the first or second handle may be attached to the first and/or second handles 105 and 115. In some embodiments, once the handles 105 and 115 have been coupled, connected, and/or are communicating with the user's computing device, the Elastic Band Bicep Curl exercise may be selected from a list of exercises presented on the exercise and therapy software application installed on the user's computing device. Once the exercise has been triggered and/or initiated on the user's computing device, the sensors (gyroscopes, magnetometers, accelerometers, and/or pressure sensors) in the handles 105 and 115 may be initialized and/or activated. In some embodiments, during a user's performance of the Elastic Band Bicep Curl, several three-dimensional angular readings, parameters, measurements and/or data (as well as tension readings, parameters, measurements and/or data) may be recorded and/or stored in memory devices of the exercise and therapy device, the user computing device and/or database server or cloud-based servers. In some embodiments, the received tension and/or three-dimensional angle measurements, parameters, and/or data received from the sensors in the exercise and therapy apparatus may be plotted or displayed in the software application by computer-readable instructions executable by the one or more processors on the user computing device and/or the database server (or cloud-based servers).

In some embodiments, tension in the cables or straps of the handles, grip force, and the user's power output may be measured and utilized as described in use case 1. In some embodiments, measurements from pressure sensors and/or strain gauges embedded in the cables or straps of the handles may be recorded, transmitted, processed and plotted on the software application to inform the user, trainer, and/or therapist of the tension in the cables or straps. The tension in the cables or straps may indicate the user's input force or load against a resistance band and/or mass during a performance and/or exercise. In some embodiments, the user's input force derived from the tension in the cables or straps may indicate muscular stability and functionality during a repetition should the tensile measurement profile remain constant, linearly increasing/decreasing, or progressively increasing/decreasing. The one or more gyroscopes, accelerometers, and magnetometers may generate three-axis angular position measurements, parameters and/or data to be used to ensure the handles 105 and 115 remain parallel to the ground surface throughout the Elastic Band Bicep Curl exercise as in prior use cases. In some embodiments, the measurements, parameters, and/or data may be recorded, transmitted, processed, and plotted using computer-readable instructions executable by the one or more processors of the user computing device (e.g. software application) as in stated in prior use case examples.

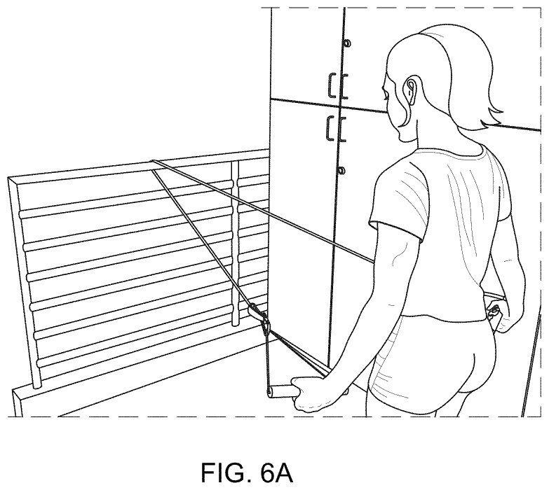

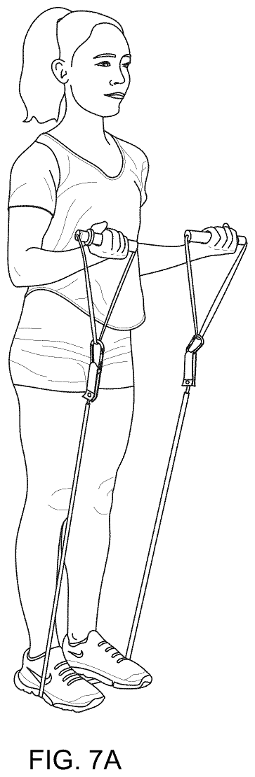

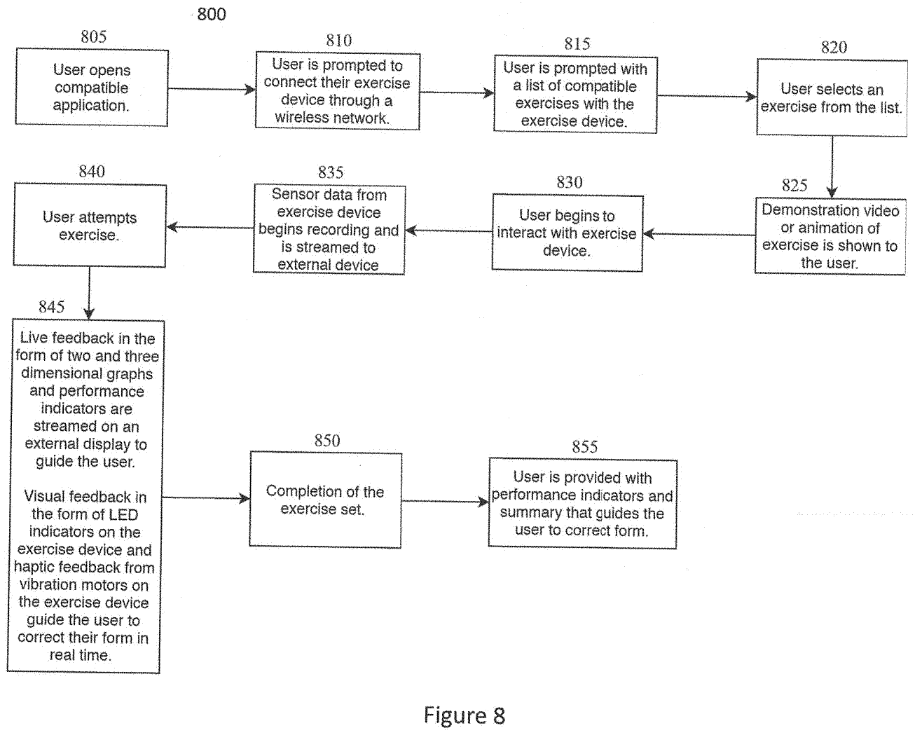

FIG. 8 illustrates a flowchart of a user interacting with an exercise and therapy device along with an exercise and therapy software application according to some embodiments. In embodiments, a user will be operating a user computing device. In some embodiments, in step 805, computer-readable instructions executable by one or more processors on the user's computing device may initiate an exercise and therapy application. In some embodiments, in step 810, the executable computer readable instructions (e.g., the exercise and therapy software application) may prompt a user to connect with an exercise and therapy device (that is either electronically connected or coupled to the user computing device or within communication range of the exercise and therapy device). In some embodiments, in step 815, in response to a user selecting to interact with an exercise and therapy device, the executable computer-readable instructions may display a list of exercises that may be performed in association or connection with the exercise and therapy device. In embodiments, the executable computer-readable instructions (software application) may identify a type of exercise and/or therapy device and may present exercise associated with the exercise and therapy device.

In some embodiments, in step 820, a user may select an exercise to be performed with the exercise and therapy device. In some embodiments, in step 825, the executable computer-readable instructions may communicate a demonstration video and/or animation to the display of the user computing device to illustrate and/or show how the exercise should be properly performed. In some embodiments, in step 830, a user may begin the selected exercise utilizing the exercise and therapy device. In some embodiments, in step 835, sensors associated with the selected exercise may captured measurements, parameters and/or data during the exercise. In some embodiments, computer-readable instructions executable by one or more processors or controllers of the exercise and therapy device may communicate, via one or more wireless communication transceivers, to the user computing device. In some embodiments, in step 840, a user continues to perform the selected exercise utilizing the exercise and therapy device.

In some embodiments, in step 845, computer-readable instructions executable by one or more processors on the user computing device may analyze the received sensor parameters, measurements and/or data and compare the received sensor parameters, measurement and/or data against prespecified thresholds and/or ranges to determine if the user is performing the exercise correctly. In some embodiments, the computer-readable instructions executable by one or more processors on the user computing device may generate and present, in real time (e.g., while exercise is being performed) on a display of a user computing device, two- or three-dimensional graphs or representations and/or indicators of a user's performance in completing the exercise in the proper fashion. In some embodiments, computer-readable instructions executable by one or more processors on the user computing device may communicate (e.g., via wireless transceivers in the user computing device and the exercise and therapy device) representations, indicators, and/or parameters on how well the user is performing the selected exercise. In some embodiments, computer-readable instructions executable by one or more processors and/or controllers on the exercise and therapy device may receive the representations, indicators and/or parameters and generate signals, messages and/or instructions, based at least in part on the representations, indicators and/or parameters and communicate the signals, messages and/or instructions to LED indicators on the exercise and therapy apparatus (e.g., for visual feedback to users) and/or vibration motors on the exercise and therapy apparatus (for haptic or touch feedback). In some embodiments, in step 850, the user may complete the selected exercise. In some embodiments, in step 855, the computer-readable instructions executable by one or more processors on the user computer may present, on a display of the user computing device, performance indicators and/or summary results based at least in part on the sensor parameters, measurements and/or data received from the exercise and therapy device during the exercise.

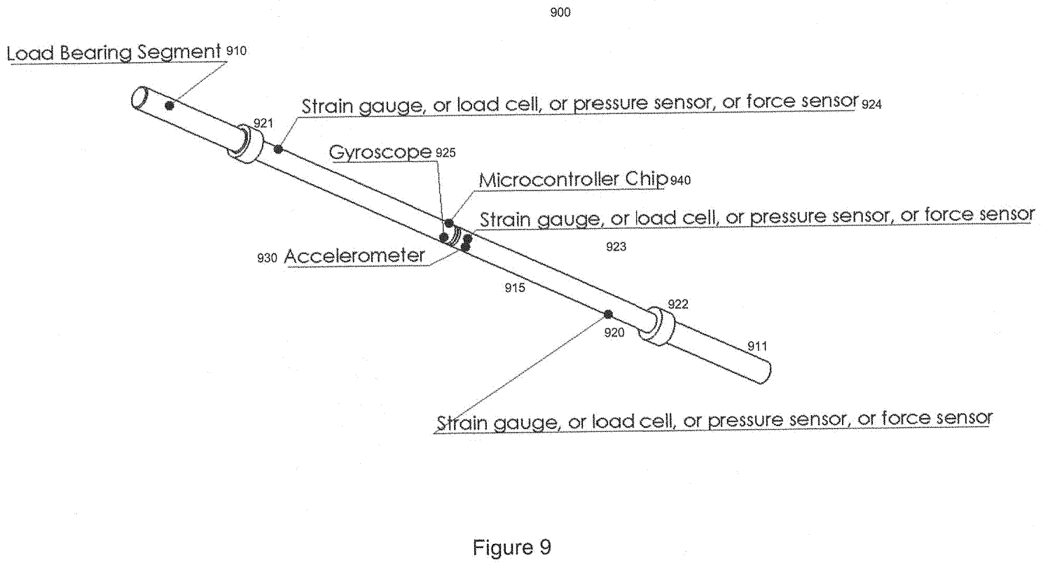

FIG. 9 illustrates location of components of an intelligent barbell according to some embodiments. In some embodiments, the intelligent barbell 900 may comprise a first end section or first load bearing segment 910, a second end section or second load bearing segment 911, and/or a central barbell section 915. In some embodiments, the central barbell section 915 may include one piece or may include multiple pieces. The components and assemblies (e.g., sensors, chips, gauges) may operate in a similar fashion to the components and assemblies described in the intelligent exercise and therapy device illustrated in FIGS. 1A-1E and FIG. 2. In some embodiments, the load bearing segments 910 or 911 may not include any components or assemblies and may be utilized to place weights on. In some embodiments, the load bearing segments 910 or 911 may be connected to the central barbell section 915 via connectors or connecting assemblies 921 and 922. In some embodiments, the intelligent barbell 915 may comprise one or more accelerometers 930, one or more gyroscopes 925, one or more strain gauges 920, 923, and 924 and/or one or more microcontroller chipsets 940. In some embodiments, one or more strain gauges 920 may be placed on an end of the central barbell section 915 which is closest to connector 922 and load bearing segment 910. In some embodiments, one or more strain gauges 924 may be placed or located on an end of the central barbell section 915 which is closest to connector 921 and load bearing segment 911. In some embodiments, the one or more strain gauges 920,923, and 924 may be a strain gauge, a load cell, a pressure sensor or a force sensor or any combination thereof. In some embodiments, the one or more strain gauges 920,923, and 924 may be placed next to load bearing segments 910 and 911, respectively, because this is where a user or patient may place their hands in order to complete an exercise or therapy routine. In some embodiments, the one or more strain gauges 920 and 923 may be placed next to load bearing segments 910 and 911, respectively, because weights will be placed onto the load bearing segments 910 and 911 and the gauges or sensors should be close to the weights that are creating forces. In some embodiments, the one or more accelerometers 930, one or more gyroscopes 925 and/or the one or more microcontroller chipsets 940 may be positioned or located near the middle of the central barbell section 915. In some embodiments, the one or more accelerometers 930 and/or the one or gyroscopes 925 may be positioned near the middle because a middle portion of the intelligent barbell may give a more accurate estimate of speed, velocity or movement of the barbell as compared to the ends because the ends of the intelligent barbell 900 may be subject to more forces. In addition, if there are issues with a user or patient completing an exercise with one hand or side different than the other, having the one or more gyroscopes 925 or accelerometers 930 near a middle area may allow the one or more gyroscopes 925 or accelerometers 930 to identify that the two sides are not in balance and the intelligent barbell is not being operated in a level or straight position. In some embodiments, the one or more microcontroller chipsets 940 may be positioned or located in a middle area of the center barbell section 915 in order to minimize any movement or load being placed on the one or more microcontroller chipsets. In other words, the middle area of the center barbell section 915 may be more stable than the outer edges of the center barbell section and/or the load bearing segments 910 and 911. In some embodiments, one or more strain gauges, load cells, pressure sensors and/or force sensors 923 may be placed in a middle of the center barbell section 915 in order to measure pressure forces in a middle of an intelligent barbell. In some embodiments, the one or more microcontroller chipset 940 may comprise one or more controllers or processors, one or more memory devices, and/or one or more wireless communication transceivers (as described in FIGS. 1A-1E, and FIG. 2). In addition, in some embodiments, computer-readable instructions may be stored in the one or more memory devices and/or may be executable by the one or more controllers or processors in order to operate certain components and/or assemblies of the intelligent barbell.

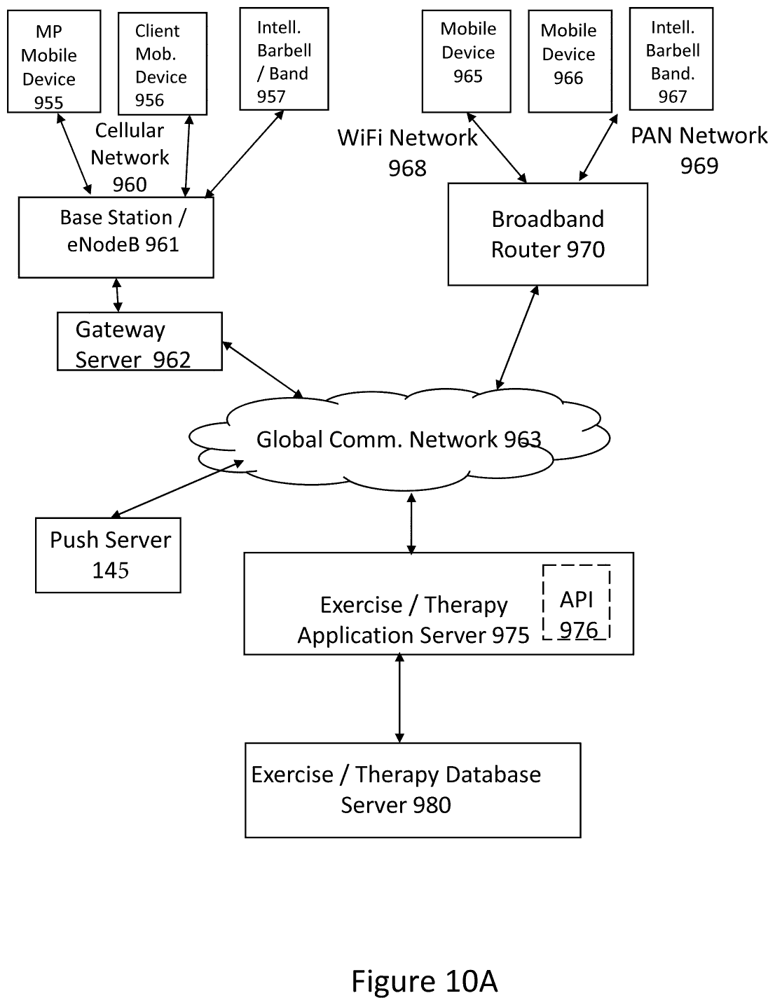

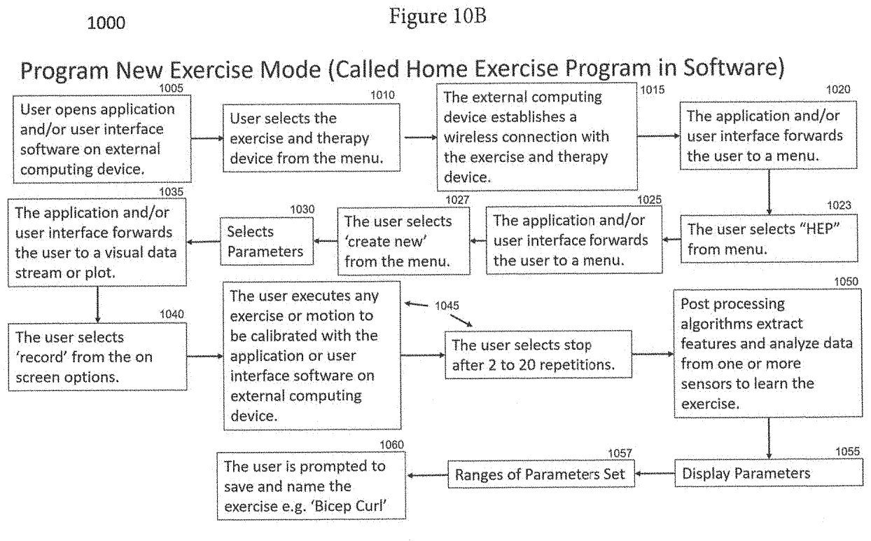

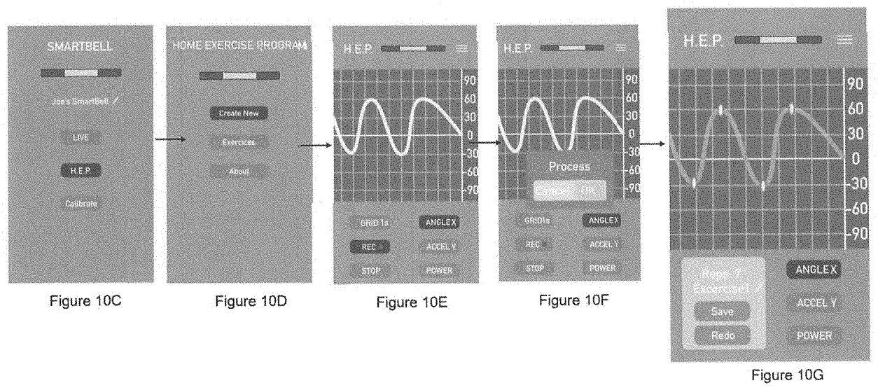

FIG. 10A illustrates an intelligent exercise or therapy software system according to some embodiments. FIG. 10A illustrates a communication and computer network system 900 that implements one or more embodiments of the intelligent exercise or therapy system. In some embodiments, the intelligent exercise or therapy system 900 includes a plurality of mobile communication or computing devices 955, 956, 957, 965, 966 and 967 are that may be coupled to a global communication network 963, such as the Internet. In some embodiments, the mobile communication or computing devices (or “mobile devices”) may be carried and operated by a user. Included in these are the intelligent barbells or intelligent exercise bands 957 and 967 described herein. In some embodiments, the mobile communication devices 955, 956, 965 and 966 may be smartphones, cellular phones, wearable computing devices, laptop computing devices and/or tablet computing devices. In some embodiments, the mobile communications devices 955, 956, 965 and 966 and the intelligent barbells or intelligent exercise bands 957 and 967 may be able to communicate with one another using known communication methods such as wireless telephony, radio, satellite, cellular systems (e.g., GSM, CDMA, 3G, 4G, 5G and so on), wireless LANS, personal area networks, or other similar systems, although not all of the mobile communication devices mobile communications devices 955, 956, 965 and 966 and the intelligent barbells or intelligent exercise bands 957 and 967 may have these features. In some embodiments, the medical professional (e.g., physical therapist, doctor, nurse practitioner or nurse) may have mobile communication or computing devices 955 or 965 to interface with the intelligent exercise or therapy system 900. In some embodiments, the clients or patients may have mobile communication or computing devices 956 and 966 that interface with the intelligent exercise or therapy systems 900 and/or may interface with the intelligent barbells and/or intelligent exercise bands 957 and 967.

In some embodiments, a server computer may be an intelligent exercise and therapy application server 975, which executes computer-readable instructions and runs an intelligent exercise and therapy system process or software (or application software). In some embodiments, the computer-readable instructions may be executable by one or more processors on the intelligent exercise and therapy application server 975. In some embodiments, the intelligent exercise and therapy system process executing on the intelligent exercise and therapy application server 975 may control various data objects relating to the exercise and/or therapy parameters, characteristics and/or information relating to the users of the intelligent barbells and/or exercise bands 957 and 967. In some embodiments, parameters, characteristics and/or information with respect to intelligent exercise and therapy system users (and/or intelligent barbells or exercise bands 957 or 967 may be stored in a database server 980 (e.g., a Rate & See database server).

In some embodiments, as is discussed below, patients and/or clients using the intelligent barbells and/or exercise bands 957 and 967 may desire or wish to utilize the equipment and to monitor performance with respect to assigned exercises and/or therapies (as well as to provide feedback to medical professionals) including physical therapists who may not be available on site. Because users of the intelligent barbells and/or exercise bands 957 and 967 as well as the medical professionals and their associated mobile communication and computing devices 955 and 965 may be transitory and on the go or engaging in some other physical or online activity, there needs to be an online system for which these medical professionals and/or patients or clients may interact with the software as well as with each other. In some embodiments, parameters and/or characteristics of users may include a profile of each user, and the preferences of each user.

In some embodiments, each user who desires to participate in the intelligent exercise and therapy software and system may pay a subscription or membership-type fee in order to participate. In some embodiments, one or more users, patients and/or medical professionals may register with the intelligent exercise and therapy software system or process by interfacing with the intelligent exercise and therapy application server 975 and providing preferences and other relevant information relating to the user, patient and/or medical professionals. In some embodiments, access may be free. In some embodiments, entered or supplied parameters, characteristics or preferences for each user, operator or physical therapist may be stored in one or more database servers 980 (e.g., intelligent exercise and/or therapy database servers), which are associated with the intelligent exercise and/or therapy application servers 975. In some embodiments, parameters, characteristics and/or preferences may be stored and/or are organized in the database servers in, for example, user profiles, user provider parameters, characteristics or preferences. In some embodiments, information and characteristics about movie titles may be stored in one or more intelligent exercise and/or therapy database servers 980.

In some embodiments, the mobile communication or computing devices 955, 956, 965, and 966 and/or intelligent barbells and/or exercise devices 957 and 967 may communicate with the global communications network 963 and the application servers 975 and/or database servers 976 via a number of communications methods. In some embodiments, as is illustrated by the mobile communication and computing devices 955 and 956, and/or intelligent barbells and/or exercise bands 957, may communicate through a cellular network 960 to base stations or eNodeBs 961 and/or gateway servers 962 and then to the global communication network 963. In this illustrative embodiment, the mobile communication and computing devices 955 and 956, and/or intelligent barbells and/or exercise bands 957 may be utilizing the cellular network to communicate with the intelligent exercise and therapy application servers 975 and/or intelligent exercise and therapy database servers 980.

In some embodiments, a mobile communication or computing devices (e.g., such as mobile devices 965 and 966) and/or intelligent barbell and/or exercise bands 967 may communicate with the global communication network 963 utilizing a WiFi (or wireless local area) network and/or a broadband router 970. In embodiments, the mobile communication devices (e.g., such as mobile devices 965 and 966) may communicate with the global communication network 963 via a personal area network (e.g., Bluetooth network) 969 and/or one or more broadband routers 970.

In some embodiments, each of the mobile communication devices 955, 956, 965 or 966 may comprise one or more processors and/or one or more memory devices. In some embodiments, computer-readable instructions executable by one or more processors may operate and initiate the intelligent exercise or therapy application software and may communicate with the intelligent exercise or therapy application server 975 and/or the intelligent exercise or therapy database server 980. In some embodiments, the computer-readable instructions executable by one or more processors may facilitate establishment and management of user's, patient's and/or medical professional's (e.g., therapists) account on the intelligent exercise or therapy application server 975 by providing a comprehensive interface to the intelligent exercise or therapy system databases 980 and processes provided on the intelligent exercise or therapy application server 975.

In some embodiments, the mobile communications devices 955, 956, 965 or 966 may interface with the intelligent exercise or therapy application servers 145 through a web server. In some embodiments, the web server may be a server or process that stores data in the form of web pages and transmits these pages as Hypertext Markup Language (HTML) files over the global communication 963 to the mobile communications devices 955, 956, 965 or 966. In some embodiments, the one or more mobile devices typically run a web browser program to access the web pages served by the web server and any available content provider or supplemental server that may also be coupled to the network.

As shown in FIG. 10A, the intelligent therapy and exercise application server computing devices 975 may execute a server-side intelligent therapy or exercise system process. In some embodiments, an application programming interface (API) 976 may allow users with mobile devices 955, 956, 965, or 966 to utilize browsers to communicate and/or login to the intelligent exercise or therapy application server 975. In some embodiments, the application server computing devices may comprise one or more processors and one or more memory devices. In embodiments, computer-readable instructions executable by the one or more processors may execute the intelligent exercise or therapy system process. In some embodiments, the one or more mobile communications devices 955, 956, 965, or 966 may run or execute a client-side version of the intelligent exercise or therapy system program, and/or they may access executable program components over the global communications network 963, such as through a web browser.

In some embodiments, data, preferences, previous measurements, previous therapy or exercise routines, characteristics and/or parameters for any of the users, patients, medical professionals and/or therapists may be stored both locally (e.g., mobile communications devices 955, 956, 965, or 966) and/or in remote computing devices (e.g., such as the 955, 956, 965, or 966 application server 975 and/or the movie title rating database server 980). In some embodiments, the prior measurements and/or parameters may include acceleration measurements, velocity measurements, the three-axis angular position measurements, power output measurements or parameters, as well as other relevant measurements. In some embodiments, the favorite or prior exercise routines may include tricep extension, rows, internal and external rotation, lay pull down for upper body, bilateral external rotation, side stepping plantar flexion, doris flexion, inversion, and eversion.

In some embodiments, the data, preferences, previous exercise or therapy measurements, previous therapy or exercise routines, characteristics and/or parameters may be stored on a separate content provider computer of the one or more database servers 980 (e.g., there may be a designated server). In some embodiments, although the one or more database servers 980 may be shown coupled to the one or more application servers 975, it should be noted that content data (e.g., data, preferences, previous exercise or therapy measurements, previous therapy or exercise routines, characteristics and/or parameters) may be stored in one or more different physical locations. In some embodiments, a intelligent exercise or therapy system process may also include a database manager program that manages the different databases stored in the one or more intelligent exercise or therapy database servers 980, which may be organized as separate databases, portions of a single database, or any other logical structure appropriate for storing the data, preferences, previous exercise or therapy measurements, previous therapy or exercise routines, characteristics and/or parameters).

As illustrated in FIG. 10A, the one or more database servers 980 stores user, patient, medical professional and/or therapist data, preferences, previous exercise or therapy measurements, previous therapy or exercise routines, characteristics and/or parameters. In some embodiments, the data, preferences, previous measurements, previous therapy or exercise routines, characteristics and/or parameters may relate to each user of mobile communications devices 955, 956, 965, or 966, and may include basic information, such as the username for the user, the password or the user and/or an email address. In embodiments, for example, a user, patient, medical professional or therapist, when creating an account on the intelligent exercise or therapist system, may provide an invite-code, which may have been communicated to the user through a messaging service, such as email and/or SMS messaging.

In some embodiments, the one or more database servers 980 may also store graphic and/or image information related to each user, patient, medical professional or therapist such as any other associated images or videos of exercise or therapy routines, threshold information and/or graphical information (e.g., such as graphs of desired ranges for measurements or parameters in exercise and/or therapy routines). In some embodiments, the one or more database servers 980 may also request a user's full name, age and/or their gender.

FIG. 10B illustrates a flowchart of a new exercise mode for the intelligent exercise or therapy software application according to some embodiments. Please note as mentioned before that the applicant refers to an intelligent exercise software application below in the specification, but the embodiments described herein also apply to an intelligent therapy software application, intelligent therapy devices and/or intelligent therapy routines. The steps outlined herein may be performed in any order. In some embodiments, some of the steps may not be performed for any of the flowcharts described herein. In some embodiments, the intelligent exercise software application initially may not have any exercises established or set up for a user to perform. Thus, a trainer, user and/or operator may need to establish exercise routines for intelligent exercise devices that communicate with and/or associated with the intelligent exercise software application. In some embodiments, the program new exercise mode of the intelligent exercise software application may allow a user or operator to establish one or more exercise routines using the intelligent barbell and/or ranges of expected measurements, metrics and/or parameters used therewith. Accordingly, in some embodiments, the program new exercise mode may allow for customized exercise routines for the users or operators, which provides the users or operators with a personalized exercise tool and/or routine.

Intelligent Exercise User Interface—In some embodiments, in step 1005, a user or operator may open or initiate an intelligent exercise software application on a mobile communication device, a desktop computing device or a wearable computing device, and an intelligent exercise software application user interface may appear on a screen of one of the computing devices described above. In some embodiments, in step 1010, a user or operator may select an exercise device from a menu of the intelligent exercise software application that is running on the computing device. In some embodiments, a device menu may include options to select, for example, an intelligent barbell, an intelligent exercise band, and/or other devices. FIG. 10C illustrates a menu after the user or operator has selected the exercise device (e.g., in this case an intelligent barbell identified as either Smartbell or Joe's Smartbell). In some embodiments, in step 1015, the mobile communication device, desktop computing device or a wearable computing device (e.g., computing device) may establish a wireless communication connection or session with the selected device. In some embodiments, this may be a Bluetooth wireless communication session, a WiFi communication session and/or a cellular communication session. In some embodiments, in step 1020, in response to the wireless communication session being established, the intelligent exercise software application may present the user or operator with a number of different operating modes for the intelligent barbell and intelligent exercise software application (e.g., 1) establish new program or Home Exercise Program (HEP) mode; 2) a live mode, and/or 3) a program or calibration mode). FIG. 10C illustrates a menu including a live mode, a HEP mode, and/or a calibrate or program mode of the intelligent exercise software application. In some embodiments, in step 1023, a user or operator may select a HEP or establish new program mode in order to create or establish exercise routines and/or expected measurements, parameters and/or metrics associated therewith.

In some embodiments, in step 1025, the intelligent exercise software application may present the user with options (such as “Create New,” “Exercises, and/or About) in order to allow users to establish new exercises or therapy routines for the users or operators. In some embodiments, FIG. 10D illustrates the exercise selection menu of the intelligent exercise software application. In some embodiments, in step 1027, a user or operator may select “a create new” button or menu in order to establish a new customized exercise or routine for the intelligent barbell.

In some embodiments, the user or operator may select the exercise routine after the user or operator has selected to create a new exercise. In some embodiments, the user or operator may select 1030 measurements to be recorded or obtained for the new exercise routine as well as metrics and/or parameters to be calculated for the new exercise routine that are based at least in part recorded measurements. In some embodiments, the user or operator may also select what is to be measured during the created exercise and/or what parameters and/or metrics may be calculated. In some embodiments, the intelligent exercise software application may have default measurements that are automatically measured during the created exercise as well as parameters and/or metrics that are automatically calculated.

In some embodiments, for example, the parameters or metrics that may be calculated for each exercise may be 1) the levelness of the intelligent barbell (e.g., calculated using measurements from the one or more accelerometers and/or measurements from the one or more gyroscopes); 2) the power output by the user of the intelligent barbell (e.g., calculated using a deflection measurement from the one or more strain gauges, measurements from the one or more accelerometers, and/or measurements from the one or more gyroscopes); 3) load from a weight on the intelligent barbell (e.g., calculated using a deflection measurement from the one or more strain gauges, measurements from the one or more accelerometers, and/or measurements from the one or more gyroscopes); and/or 4) force input from the user or operator (e.g., calculated using a deflection measurement from the one or more strain gauges, measurements from the one or more accelerometers, and/or measurements from the one or more gyroscopes). In some embodiments, the parameters or metrics that may be measured for each exercise may also be 5) x-direction, y-direction and/or z-direction accelerations, velocities and/or displacements; and/or 6) grip locations of a user or operator's hand during the exercise.

In some embodiments, in step 1035, in response to the selected exercise and/or the selected parameters or metrics for the selected exercise the intelligent exercise software application may generate one or more visual data streams or plot for the newly created exercise which may be displayed on the user interface. Because the user or operator has not performed the created exercise routines, the graphs or plots may not include any data and/or measurements and may just show possibility of different labels for the axis (e.g., time versus the metric to be measured). In some embodiments, the intelligent exercise software application may allow a user to select which metric or parameter may be displayed on the graph (e.g., in FIG. 10E, the user or operator may select to display an x-direction or x-angle measurement, a y-angle measurement and/or a user power metric or parameter).

In some embodiments, in step 1040, a user or operator may select a record and/or capture option on the intelligent exercise software application menu. In some embodiments, this means that intelligent exercise software application may be initiated to capture the selected measurements and/or to calculate the selected metrics and/or parameters for the selected created exercise.

In some embodiments, the user or operator may perform a number of repetitions of the selected exercise and the intelligent exercise software application may store 1045 captured measurements and calculated metrics or parameters for the selected new exercise in one or more memory devices of intelligent barbell and/or the computing device (and/or server computing devices). In some embodiments, the user may execute any exercise or motion to be calibrated with the application or user interface software and repeat the exercise or motion. In some embodiments, the user or operator may then stop the selected exercise routine and provide an input to the intelligent exercise software application that the selected exercise has been stopped.

In some embodiments, the intelligent exercise software application may analyze the captured measurements from the sensors, devices and/or assemblies of the intelligent barbell and may calculate 1050 metrics and/or parameters for the created exercise routine. In these embodiments, the metrics and/or parameters may be the levelness of the intelligent barbell; the power output by the user of the intelligent barbell; load from a weight on the intelligent barbell; force input from the user or operator; x-direction, y-direction and/or z-direction accelerations, velocities and/or displacements; and/or grip locations of a user or operator's hand during the exercise.

In some embodiments, in step 1055, the intelligent exercise software application may then generate and/or display the calculated metrics and/or parameters on graphs or visual displays. In some embodiments, the user may identify ranges of measurements and/or ranges of metrics and/or parameters 1057 that are acceptable for created exercise routine. These may also be referred to as envelopes of acceptable measurements and/or acceptable calculated metrics or parameters. In some embodiments, the intelligent exercise software application may automatically determine acceptable measurements for the created exercise routine and/or acceptable calculated metrics or parameters (e.g., the intelligent exercise software application may add a 5-10% increase and/or subtract a 5-10% decrease from the average captured measurements, metrics and/or parameters). In some embodiments, the determined ranges of measurements, parameters and/or metrics may be stored for the created exercise routine in one or more memory devices of the intelligent barbell and/or the computing devices running the intelligent exercise software application. FIG. 10E illustrates an x-angular displacement of an intelligent barbell during the selected exercise according to embodiments. FIG. 1OF illustrates a screen requesting that the user or operator select whether or not to process the captured metrics and/or parameters for the selected exercise. FIG. 10G illustrates a screen that is displayed after the set number of repetitions have been performed and providing a user or operator with the option of having the user or operator perform the selected exercise again (or redo the selected exercise) to obtain different data or letting the user or operator to store the captured metrics and/or parameters for the selected exercise.

In some embodiments, in step 1060, the intelligent exercise software application may receive 1060, from a user or operator, a name of the created exercise and may store this in the one or more memory devices. For example, some example exercises that may be created are a bicep curl, an intelligent barbell dead lift, an elastic band low row, an elastic band squat or an intelligent barbell squat, and/or an elastic band external rotation with abduction. In some other embodiments, when a user initially selected the exercise device 1010, the user or operator may also select a specific exercise routine as the exercise routine that may be created (e.g., such as a bicep curl, a barbell dead lift, an elastic low row, an elastic band squat, and/or a band external rotation with abduction).

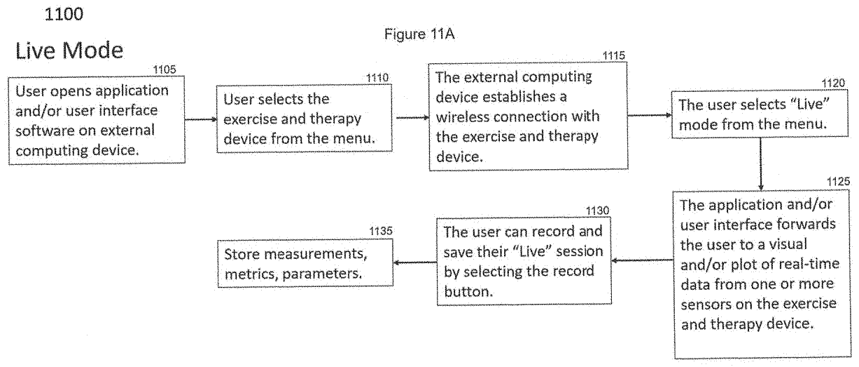

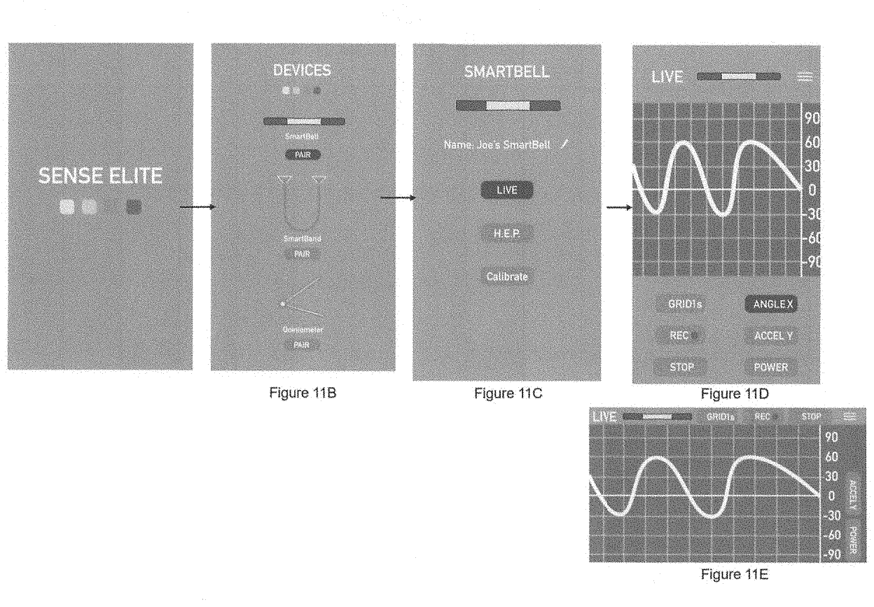

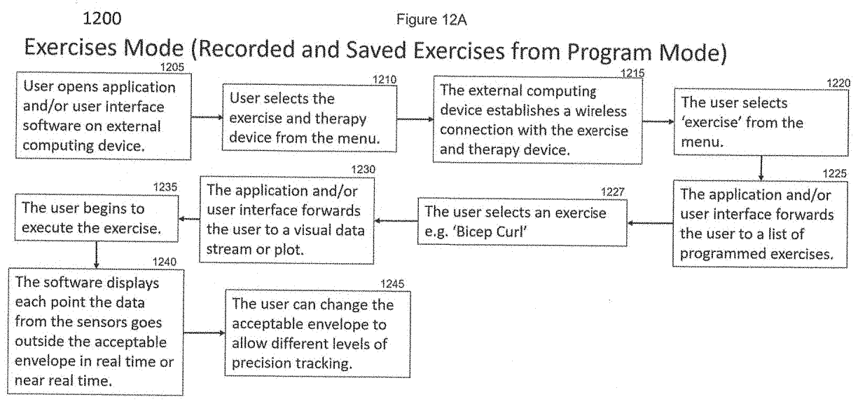

FIG. 11A illustrates a flowchart of an intelligent exercise software application live mode according to some embodiments. In some embodiments, in step 1105, a user or operator may open or initiate an intelligent exercise software application on a mobile communication device, a desktop computing device or a wearable computing device, and an intelligent exercise user interface may appear on a screen of one of the computing devices described above. In some embodiments, in step 1110, a user or operator may select an exercise device and/or an exercise routine from a menu of the intelligent exercise software application that is running on a computing device. In some embodiments, a device menu may include options to select, for example, an intelligent barbell, an intelligent exercise band or smart band, a goniometer, and/or other devices. FIG. 11B illustrates a menu generated by the intelligent exercise software application to allow a user or operator to select an exercise device according to some embodiments. In some embodiments, the intelligent exercise software application may generate a menu that allows a user or operator to select one of the existing exercise routines after the user or operator has selected the exercise device (e.g., in this case an intelligent barbell identified as either SmartBell or SmartBand). In some embodiments, in step 1115, the mobile communication device, desktop computing device or a wearable computing device (e.g., computing device) may establish a wireless communication connection or session with the selected exercise or therapy device. In some embodiments, this may be a Bluetooth wireless communication session, a WiFi communication session and/or a cellular communication session. In some embodiments, in step 1120, in response to the wireless communication session being established, the intelligent exercise software application may present the user or operator with a number of different operating modes (e.g., establish new program or Home Exercise Program (HEP) mode; a live mode, and/or a program mode). FIG. 11C illustrates a menu including a live mode, a HEP mode, and/or a calibrate or program mode. In some embodiments, in step 1123, a user or operator may select a live mode (or a live exercise mode). In this mode, the intelligent exercise software application may be able to record their specific measurements for the selected exercise routine. In some embodiments, the intelligent exercise software application may generate and/or present 1125 a user or operator with one or more graphs or visual plots that may be utilized during the exercise routine for the users to keep track on their progress and/or performance. In some embodiments, the user or operator may select and/or the intelligent exercise software application may receive the selection of the live mode 1130. In some embodiments, the user or operator may begin the selected exercise. In some embodiments, in response to the live mode being selected, the intelligent exercise software application may cause measurements from the intelligent barbell sensors, assemblies or gauges to be captured and may store 1135 the captured measurements in one or more memory devices of the intelligent barbell and/or the computing devices. In some embodiments, the intelligent exercise software application may calculate metrics and/or parameters for the exercise routine based at least in part on the captured measurements, and may store the calculated metrics and/or parameters in the one or more memory devices of the intelligent barbell and/or the computing device. In some embodiments, the intelligent exercise software application may present the user's data on the generated plots and/or graphs. FIG. 11D illustrates the display of a user's x-angle displacement over a period of time utilizing the intelligent barbell for the selected exercise according to some embodiments. FIG. 11E illustrates the display of a user's y-angle displacement over a period of time utilizing the intelligent barbell for the selected exercise according to some embodiments. In some embodiments, the user or operator may stop performing the exercise routine and the intelligent exercise software application may stop recording the user or operator's measurements. In some embodiments, the intelligent exercise software application may request that the user or operator confirm that the live mode be exited or stopped. Utilizing the live mode, the user or operator may thus keep track of the user's performance of the selected exercise routine on the selected exercise device.

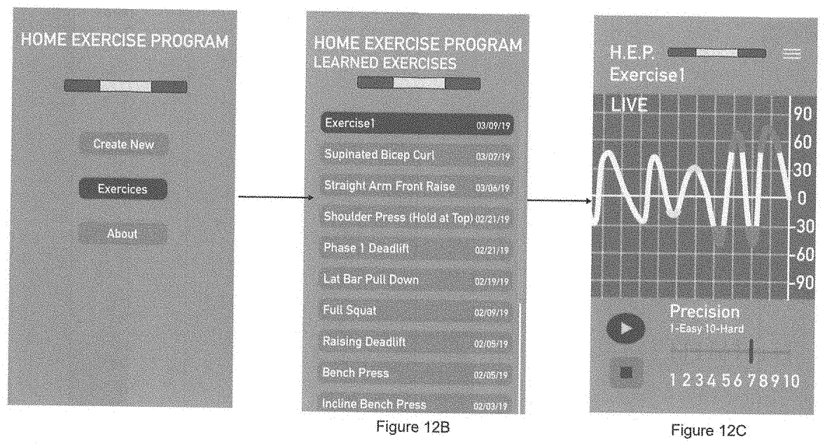

FIG. 12A illustrates a flowchart of an intelligent exercise software application exercise or calibrate mode according to some embodiments. In some embodiments, in step 1205, a user or operator may open or initiate an intelligent exercise software application on a mobile communication device, a desktop computing device or a wearable computing device, and an intelligent exercise user interface may appear on a screen of one of the computing devices described above. In some embodiments, in step 1210, a user or operator may select an exercise and therapy device from a menu of the intelligent exercise software application that is running on a computing device. In some embodiments, a device menu may include options to select, for example, an intelligent barbell, an intelligent exercise band or smart band, a goniometer, and/or other devices. In some embodiments, in step 1215, the mobile communication device, desktop computing device or a wearable computing device (e.g., computing device) may establish a wireless communication connection or session with the selected exercise or therapy device. In some embodiments, this may be a Bluetooth wireless communication session, a WiFi communication session and/or a cellular communication session. In some embodiments, in step 1220, in response to the wireless communication session being established, the intelligent exercise software application may present the user or operator with a number of different operating modes (e.g., establish new program or Home Exercise Program (HEP) mode; a live mode, and/or a exercise, program or calibration mode). In some embodiments, in step 1223, a user or operator may select an exercise, a program or a calibration mode. In this mode, the intelligent exercise software application may be able to record their specific measurements for the selected exercise routine. In some embodiments, the intelligent exercise software application may generate and/or present 1225 a user or operator a number of saved or previously created exercise routines. FIG. 12B illustrates a list of previously created exercise routines for the intelligent exercise software application according to some embodiments. In some embodiments, the intelligent exercise software application may receive 1227 the user's selection of the exercise routine. In some embodiments, the intelligent exercise software application may generate and/or present 1230 one or more graphs or visual plots that may be utilized during the selected exercise routine for the users to keep track on their progress and/or performance.

In some embodiments, the user or operator may begin to perform 1235 the selected exercise routine utilizing the selected exercise device.

In some embodiments, the intelligent exercise software application may capture measurements from the sensors, gauges and/or other assemblies of the intelligent barbell. In some embodiments, the intelligent exercise software application may calculate metrics and/or parameters based at least in part on the captured measurements.

In some embodiments, the intelligent exercise software application may display the captured measurements and/or the calculated metrics and/or parameters on the graphs and/or visual plots. In some embodiments, included in this display, may be measurements, metrics and/or parameters that are outside of an expected envelope established for the exercise routine. In some embodiments, the intelligent exercise software application may separately visually identify 1240 the portions of measurements, metrics, and/or parameters that are outside of the expected or established envelope of measurements, metrics and/or parameters (e.g., in a different color and/or intensity). In some embodiments, the display of the outlying measurements, metrics and/or parameters may occur in real time as the exercise is being performed. In some embodiments, the intelligent exercise software application may also cause the computing device to make audible warnings that the user is outside the expected envelope. In some embodiments, the intelligent exercise software application may communicate warnings to the intelligent barbell and the intelligent barbell may provide the user with haptic feedback (e.g., shake or move the intelligent barbell) or potentially visual feedback (e.g., flashing lights). In some embodiments, the user or operator may also allow a precision parameter to be set for exercise routines in the intelligent exercise software application. This may determine a percentage from average measurements, metrics or parameters that the intelligent exercise software application is willing to allow before alarms or warnings are generated. FIG. 12C illustrates a menu to allow setting of a precision parameters according to some embodiments. In FIG. 12C, a low precision parameter means that a higher percentage of variation or deviation may be allowed, whereas a high precision parameter (e.g., 10) means that a lower percentage of variation or deviation may be allowed before alarms or warnings are generated in the intelligent exercise software application. In some embodiments, the intelligent exercise software application may also allow the operator to modify 1245 the ranges of acceptable measurements, metrics and/or parameters for the selected exercise. In some embodiments, the ranges of the acceptable measurements, metrics and/or parameters may be referred to as an acceptable envelope of measurements, metrics and/or parameters. In some embodiments, the user may provide the modified envelope of measurements, metrics and/or parameters for the selected exercise routine and the intelligent exercise software application may store the modified envelope of measurements, metrics and/or parameters in the one or more memory devices of the computing devices and/or the intelligent barbell.

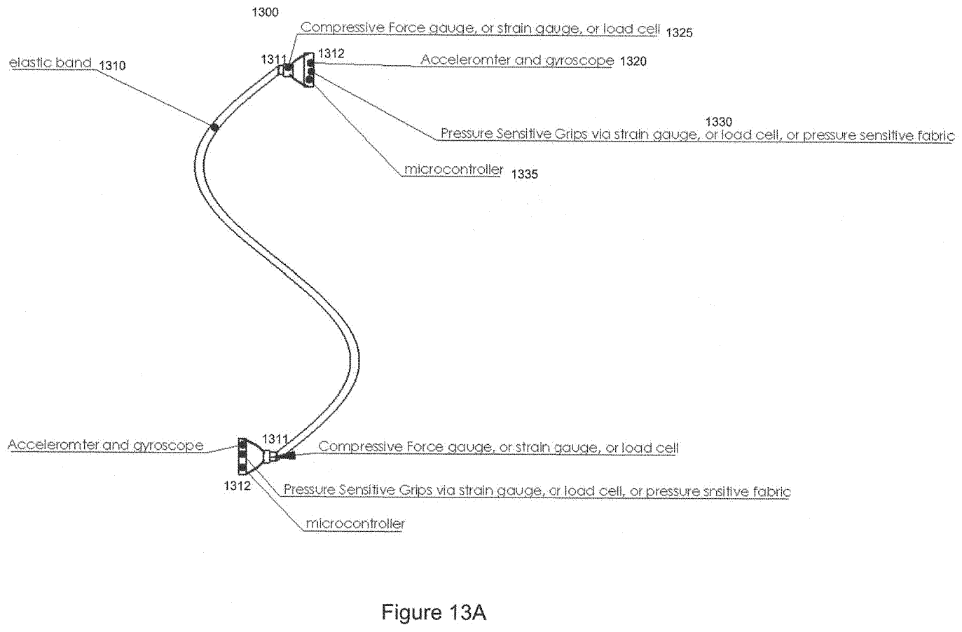

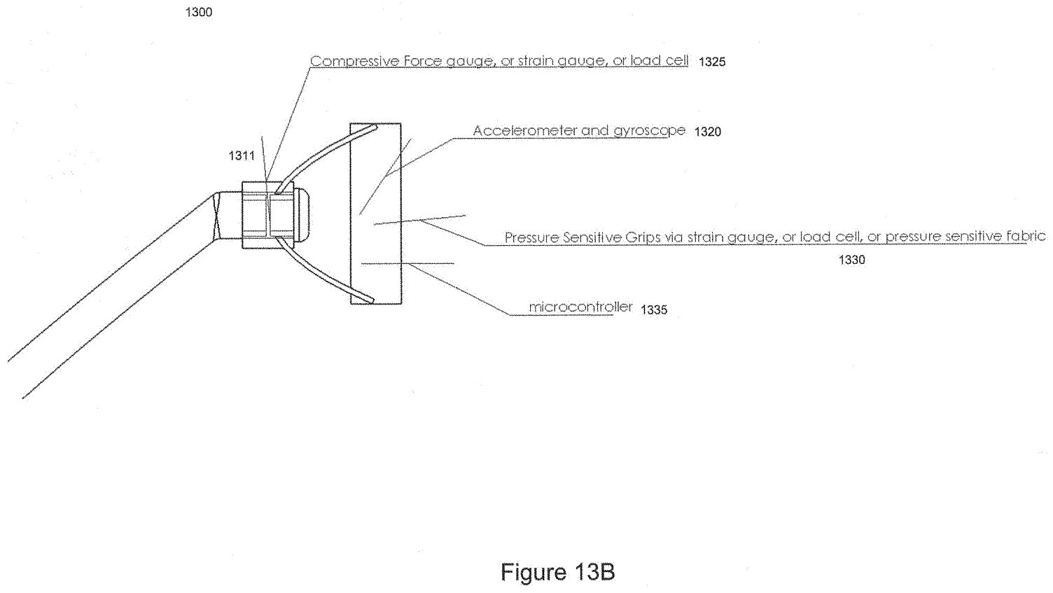

FIG. 13A illustrates an intelligent exercise band according to some embodiments. FIG. 13B illustrates a handle of an intelligent exercise band according to some embodiments. In some embodiments, the intelligent exercise band on its own may be utilized to perform exercises (e.g., such as the elastic band bicep curl) that have been discussed above. In some embodiments, the intelligent exercise band 1300 may comprise an elastic band 1310 that has two ends, where each of the two ends includes a connector or connector assembly 1311 and a handle 1312. In some embodiments, the connector assembly 1311 may connect, couple or attach the handle 1312 to the elastic band 1310. In some embodiments, a connector assembly 1311 may comprise one or more compressive force gauges 1325. In some embodiments, one or more compressive force gauges 1325 may measure force exerted by a patient or individual utilizing the intelligent exercise band for exercises. In some embodiments, rather than compressive force gauges 1325, one or more strain gauges and/or one or more load cells may be utilized to measure force exerted by the patient or individual. In some embodiments, the one or more compressive force gauges 1325 may be located on the elastic band 1310 and/or on the handles 1312. In some embodiments, the handles 1312 may be range between 4 to 10 inches. In some embodiments, the connector assembly 1311 may have a diameter between 0.1 to 4.5 inches. In some embodiments, the elastic band may range from 3 to 10 feet.

In some embodiments, the one or more handles 1312 may include one or more pressure sensitive grips utilizing strain gauges 1330, one or more accelerometers and/or gyroscopes 1320, and/or one or more microcontrollers 1335. In some embodiments, the one or more pressure sensitive grips 1312 may measure strain or force exerted by the user. In some embodiments, the one or more accelerometers and/or gyroscopes 1320 may measure a levelness of the handle or hand while exercise is being performed as well as a speed or velocity of the handle and/or hand while exercise is being performed. In some embodiments, the one or more microcontrollers 1335 (as mentioned previously) may include one or more controllers or processors, one or more memory devices and/or computer-readable instructions executable by the one or more processors to perform certain actions of the exercise band 1300 such as capturing data, parameters and/or measurements from the one or more accelerometers and/or gyroscopes 1320, the compression force gauges 1325 and/or the pressure sensitive grips 1330, processing the data and/or parameters and/or transferring the data, parameters and/or measurements to an external computing device and/or storing the data, parameters and/or measurements in the one or more memory devices. In some embodiments, the intelligent exercise band 1300 may further comprise one or more wireless transceivers (not shown) to communicate with external computing devices (e.g., to receive instructions for operation of the intelligent barbell and/or to communicate parameters, measurements and/or data to the external computing device). In other words, the intelligent exercise band 1300 may have bidirectional or two-way communication with the outside world (e.g., including one or more external computing devices).

In some embodiments, a physical therapy software application may assist in ensuring that patients are performing recommended and/or prescribed exercises at home by providing the patient with immediate and/or up to date parameters, data and/or measurements. This supply of immediate and/or up-to-date parameters, measurements and/or data allows patients to progress faster and helps the patients or clients independent. In some embodiments, the physical therapy software application allows physical therapists to provide a bridge between a clinic and/or at home exercises. In some embodiments, the physical therapy software application may allow a physical therapist to monitor the clients and/or patients when they perform exercises at home. In some embodiments, the physical therapy software application may allow clients and/or physical therapists to clearly visualize device movement along with the calculated measurements, parameters and/or data. In some embodiments, the physical therapist may provide immediate feedback through a messaging portal of the physical therapy software application to correct a patient's form and/or give positive feedback.

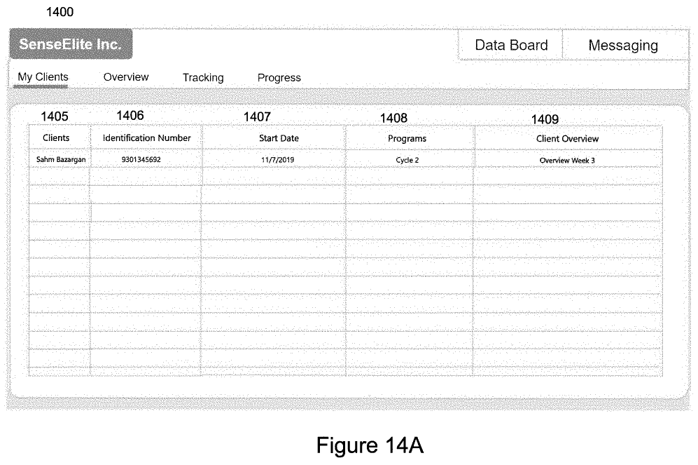

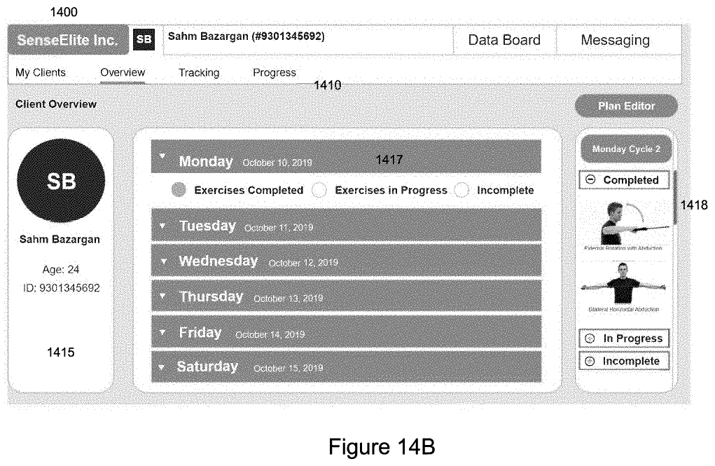

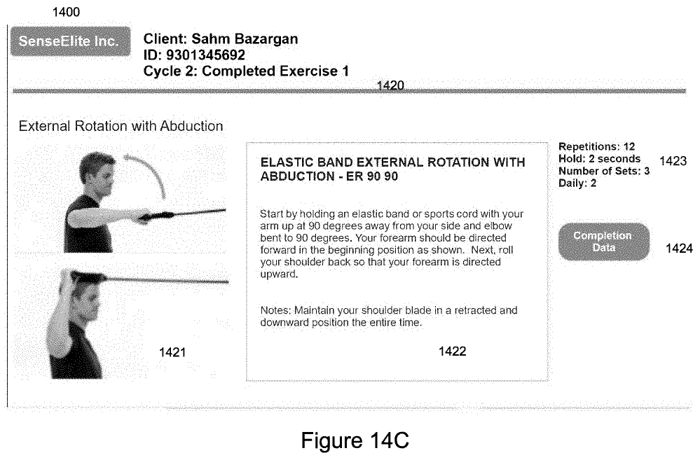

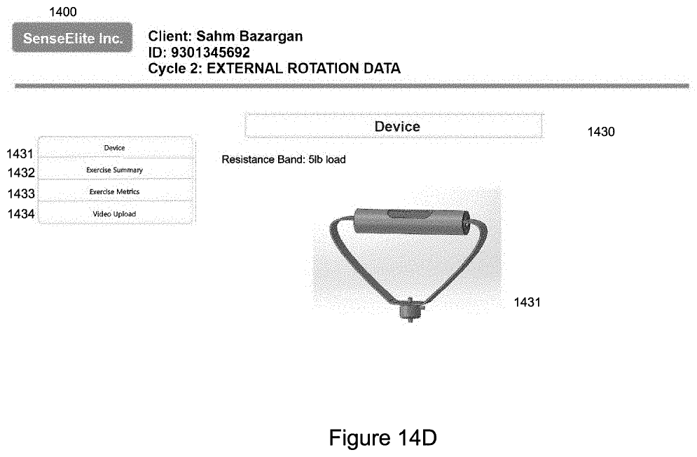

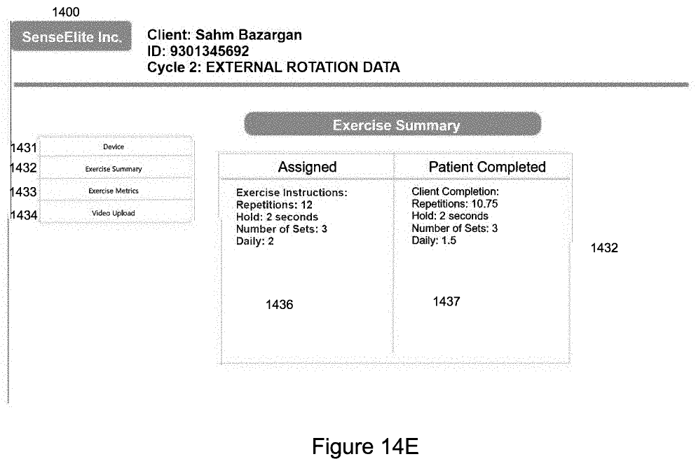

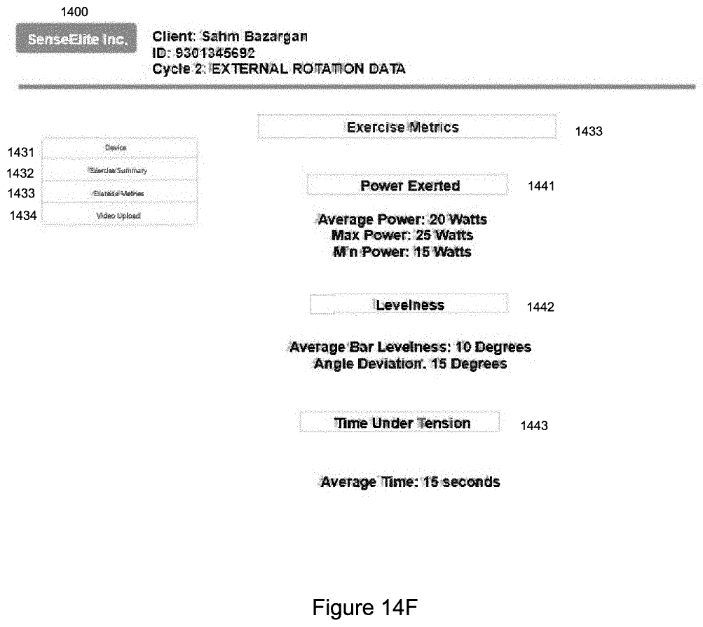

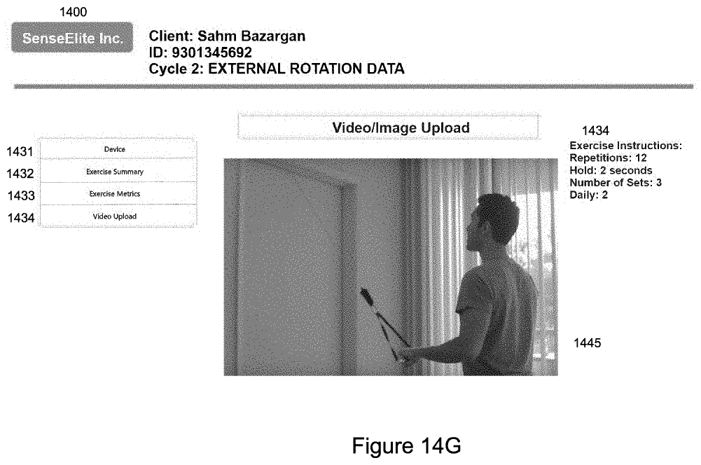

FIG. 14A illustrates a physical therapist client input screen according to some embodiments. In some embodiments, the client input screen may include a client's menu, an overview menu, a tracking menu and/or a progress menu. In the client's menu, the client's menu may include a spreadsheet including a client column 1405, an identification number column 1406, a start date column 1407, an exercise program column 1408, and/or a client overview column 1409. In some embodiments, the client's column 1405 lists clients that a physical therapist or a medical provider may have enrolled and/or assigned. In some embodiments, the identification number column may be a medical ID number or other number assigned by the medical provider. In some embodiments, the start date column 1407 may list when the patient or client initiated or started exercises. In some embodiments, the programs column 1408 may list the programs that the exercise programs that the patient is engaged in. In some embodiments, the overview column may list where a patient is in a therapy routine or program (e.g., how many weeks into a program).

FIG. 14B illustrates an overview menu of a physical therapy software application according to some embodiments. In some embodiments, the overview menu 1410 may comprise a patient submenu 1415, a daily exercise routine submenu 1417, and/or an exercise overview menu 1418. In some embodiments, the patient submenu 1415 may display a patient or client's name, an abbreviation, an age and/or a medical identification number. In some embodiments, the daily exercise routine submenu 1417 may display a week or a month. In some embodiments, after a day is selected on the daily exercise routine submenu 1417, the daily exercise routine submenu 1417 may display an option to view exercises completed, exercises in progress and/or incomplete exercises. In some embodiments, the exercise overview menu 1418 may allow a physical therapist or patient to view the exercises complete, the exercises in progress and/or the exercises that are not complete. In some embodiments, the overview menu 1410 may also allow a physical therapist to edit a rehabilitation or physical therapy plan.

FIG. 14C illustrates an exercise description screen or menu according to some embodiments. In some embodiments, the exercise description screen or menu 1420 includes an exercise illustration or display submenu 1421, an exercise detailed description submenu 1422, an exercise statistics submenu 1423 and/or a completion data submenu 1424. In some embodiments, the exercise illustration or display submenu 1421 may display individuals performing the exercise, either through images, animations and/or video and also display proper technique for performing the exercise. In some embodiments, the exercise detailed description submenu 1422 may provide details and/or information about the exercise or therapy to the patient. For example, the exercise detailed description submenu 1422 may identify positions of body parts and directions of movements that are performed in the exercise or therapy. In some embodiments, the exercise statistics submenu 1423 may detail or identify the number of repetitions to be performed for each exercise, how long the patient or client should hold the exercise for (e.g., in seconds), how many sets of repetitions that should be performed, and/or completion data for when this exercise had been previously performed.

FIG. 14D illustrates an exercise performance screen or menu according to some embodiments. In some embodiments, the exercise performance screen or menu 1430 may include four submenus: an exercise device submenu 1431, an exercise summary comparison submenu 1432, a calculated exercise metrics submenu 1433, and/or a video upload submenu 1434. FIG. 14D further illustrates an exercise device submenu 1431 and displays a handle and/or connector of an intelligent exercise band. In some embodiments, the exercise device submenu 1431 may further display the intelligent exercise band, the intelligent barbell, and/or other intelligent exercise device. In some embodiments, the exercise device submenu 1431 may provide additional details of the exercise device (e.g., that the resistance band has a 5 pound (lb) load).

FIG. 14E illustrates an exercise summary comparison submenu according to some embodiments. In some embodiments, the exercise summary comparison submenu 1432 may display an assigned exercise routine 1436 and/or a completed patient exercise routine 1437. In other words, the assigned exercise routine 1436 is what the medical professional (e.g., physical therapist) assigned to the client or patient and the completed patient exercise routine 1437 is what exercises and amount of exercises the patient actually performed. Thus, the exercise summary comparison submenu provides the physical therapist and/or medical profession (as well as the patient or client) with an up-to-date performance summary for the client.

FIG. 14F illustrates a calculated exercise metrics submenu according to some embodiments In some embodiments, a calculated exercise metrics submenu 1433 may comprise a power exerted submenu 1441, a levelness submenu 1442, and/or a time under tension submenu 1443. While these three metrics are displayed in the calculated exercise metrics submenu 1433, other metrics may be added and/or substituted into the submenu. In some embodiments, computer-readable instructions executable by one or more microprocessors of an intelligent exercise device may calculate the metrics that are displayed in the calculated exercise metrics submenu. In some embodiments, computer-readable instructions executable by one or more microprocessors of an external computing device may calculate the metrics that are displayed in the calculated exercise metrics submenu. In some embodiments, for example, the power exerted submenu 1441 may display an average power, a maximum power and/or a minimum power. In some embodiments, the levelness submenu 1442 may display an average bar levelness measurement and/or an angle deviation measurement. In some embodiments, a time under tension submenu 1443 may display an amount of time or an average time that the client or the patient has been using the exercise device.

FIG. 14G illustrates a video upload submenu according to some embodiments. In some embodiments, the video upload submenu 1434 allows a patient or client to take a video of the patient or client to upload the menu. In some embodiments, the video upload submenu 1434 may have a screen 1445 which shows the video that has been uploaded. In some embodiments, this allows the medical professional or physical therapist to see exactly how the patient or client is performing the exercise. Thus, the medical professional or physical therapist can provide feedback because not only can the physical therapist or medical professional see the calculated metrics but also actual performance of the exercise. This provides a significant advantage over other computing devices. The term “memory” or “memory device,” as used herein, generally represents any type or form of volatile or non-volatile storage device or medium capable of storing data and/or computer-readable instructions. In one example, a memory device may store, load, and/or maintain one or more of the modules described herein. Examples of memory devices comprise, without limitation, Random Access Memory (RAM), Read Only Memory (ROM), flash memory, Hard Disk Drives (HDDs), Solid-State Drives (SSDs), optical disk drives, caches, variations or combinations of one or more of the same, or any other suitable storage memory.

In addition, the term “processor” or “physical processor,” as used herein, generally refers to any type or form of hardware-implemented processing unit capable of interpreting and/or executing computer-readable instructions. In one example, a physical processor may access and/or modify one or more modules stored in the above-described memory device. Examples of physical processors comprise, without limitation, microprocessors, microcontrollers, Central Processing Units (CPUs), Field-Programmable Gate Arrays (FPGAs) that implement softcore processors, Application-Specific Integrated Circuits (ASICs), portions of one or more of the same, variations or combinations of one or more of the same, or any other suitable physical processor.

Although illustrated as separate elements, the method steps described and/or illustrated herein may represent portions of a single application. In addition, in some embodiments one or more of these steps may represent or correspond to one or more software applications or programs that, when executed by a computing device, may cause the computing device to perform one or more tasks, such as the method step.

In addition, one or more of the devices described herein may transform data, physical devices, and/or representations of physical devices from one form to another. For example, one or more of the devices recited herein may receive image data of a sample to be transformed, transform the image data, output a result of the transformation and store the result of the transformation to produce an output image of the sample. Additionally or alternatively, one or more of the modules recited herein may transform a processor, volatile memory, non-volatile memory, and/or any other portion of a physical computing device from one form of computing device to another form of computing device by executing on the computing device, storing data on the computing device, and/or otherwise interacting with the computing device.

The term “computer-readable medium,” as used herein, generally refers to any form of device, carrier, or medium capable of storing or carrying computer-readable instructions. Referrals to instructions refers to computer-readable instructions executable by one or more processors in order to perform functions or actions. The instructions may be stored on computer-readable mediums and/or other memory devices. Examples of computer-readable media comprise, without limitation, transmission-type media, such as carrier waves, and non-transitory-type media, such as magnetic-storage media (e.g., hard disk drives, tape drives, and floppy disks), optical-storage media (e.g., Compact Disks (CDs), Digital Video Disks (DVDs), and BLU-RAY disks), electronic-storage media (e.g., solid-state drives and flash media), and other distribution systems.

A person of ordinary skill in the art will recognize that any process or method disclosed herein can be modified in many ways. The process parameters and sequence of the steps described and/or illustrated herein are given by way of example only and can be varied as desired. For example, while the steps illustrated and/or described herein may be shown or discussed in a particular order, these steps do not necessarily need to be performed in the order illustrated or discussed.

The various exemplary methods described and/or illustrated herein may also omit one or more of the steps described or illustrated herein or comprise additional steps in addition to those disclosed. Further, a step of any method as disclosed herein can be combined with any one or more steps of any other method as disclosed herein.

Unless otherwise noted, the terms “connected to” and “coupled to” (and their derivatives), as used in the specification and claims, are to be construed as permitting both direct and indirect (i.e., via other elements or components) connection. In addition, the terms “a” or “an,” as used in the specification and claims, are to be construed as meaning “at least one of.” Finally, for ease of use, the terms “including” and “having” (and their derivatives), as used in the specification and claims, are interchangeable with and shall have the same meaning as the word “comprising.

The processor as disclosed herein can be configured with instructions to perform any one or more steps of any method as disclosed herein. As used herein, the term “or” is used inclusively to refer items in the alternative and in combination. As used herein, characters such as numerals refer to like elements.

Embodiments of the present disclosure have been shown and described as set forth herein and are provided by way of example only. One of ordinary skill in the art will recognize numerous adaptations, changes, variations and substitutions without departing from the scope of the present disclosure. Several alternatives and combinations of the embodiments disclosed herein may be utilized without departing from the scope of the present disclosure and the inventions disclosed herein. Therefore, the scope of the presently disclosed inventions shall be defined solely by the scope of the appended claims and the equivalents thereof.

The above disclosure is sufficient to enable one of ordinary skill in the art to practice the invention, and provides the best mode of practicing the invention presently contemplated by the inventor. While there is provided herein a full and complete disclosure of the preferred configurations of this invention, it is not desired to limit the invention to the exact construction, dimensional relationships, and operation shown and described. Various modifications, alternative constructions, changes and equivalents will readily occur to those skilled in the art and may be employed, as suitable, without departing from the true spirit and scope of the invention. Such changes might involve alternative materials, components, structural arrangements, sizes, shapes, forms, functions, operational features or the like. The invention has been described herein using specific embodiments for the purposes of illustration only. It will be readily apparent to one of ordinary skill in the art, however, that the principles of the invention can be embodied in other ways. Therefore, the invention should not be regarded as being limited in scope to the specific embodiments disclosed herein, but instead as being fully commensurate in scope with the following claims.

1. An intelligent exercise or therapy device, comprising:
one or more handles, the one or more handles comprising:
one or more accelerometers, one or more gyroscopes or directional sensors, one or more magnetometers, to capture multi-axis angular measurements, the one or more accelerometers to capture velocity or acceleration measurements;
one or more pressure sensors or strain gauges to capture grip strength measurements; and
one or more wireless communication transceivers to transmit the captured multi-axis measurements, the captured grip strength measurements and the captured velocity or acceleration measurements to a computing device; and
one or more connectors, the one or more connectors to connect the one or more handles to an end of an exercise band or an end of a barbell.

2. The intelligent exercise or therapy device of claim 1, further comprising one or more microcontrollers, the one or more microcontrollers to interface with the one or more accelerometers, gyroscopes or direction sensors; magnetometers; the one or more pressure sensors or strain gauges; and the one or more wireless communication transceivers and the one or more microcontrollers to process the captured multi-axis measurements, the captured grip strength measurements, the captured acceleration measurements or the captured velocity measurements before the measurements are transmitted by the one or more wireless communication transceivers.

3. The intelligent exercise or therapy device of claim 1, wherein the one or more connectors include one or more compression force gauges, strain gauges or load cells to capture load measurements or force applied measurements and wherein the one or more wireless communication transceivers to transmit the captured load measurements or force applied measurements to the computing device.

4. The intelligent exercise or therapy device of claim 1, the one or more accelerometers, the one or more gyroscopes or the directional sensors, the one or more magnetometers further to capture location measurements and the one or more wireless communication transceivers to transmit the captured location measurements to the computing device.

5. The intelligent exercise or therapy device of claim 1, wherein the one or more pressure sensors or strain gauges capture weight lifted parameters and the one or more wireless communication transceivers transmit the captured weight lifted parameters to the computing device.

6. The intelligent exercise or therapy device of claim 1, wherein the connector is connected to an end of an exercise band.

7. The intelligent exercise or therapy device of claim 6, further comprising another connector and another handle, the another connector to connect the handle to another end of the exercise band.

8. The intelligent exercise or therapy device of claim 1, wherein the connector is connected to an end of an intelligent barbell.

9. The intelligent exercise or therapy device of claim 4, the one or more gyroscopes or directional sensors, the one or more accelerometers, the one or more magnetometers, the one or more strain gauges and/or the one or more pressure sensors are attached to a surface of the handle.

10. The intelligent exercise or therapy device of claim one, the one or more handles further comprising one or more memory devices and computer-readable instructions executable by the one or more microcontrollers to control operation of various components of the handle of the intelligent exercise device.

11. The intelligent exercise or therapy device of claim 1, the compression sensors comprising a compressive force gauge, a strain gauge, or a load cell.

12. A method of utilizing an intelligent exercise device in performing an exercise, comprising:
a computing device, the computing device comprising:
one or more processors;
one or more memory devices;
computer-readable instructions stored in the one or more memory devices, the computer- readable instructions accessible from the one or more memory devices and executable by the one or more processors to:
establish a wireless communication connection with the intelligent exercise or therapy device, the computing device being separate from an exercise or therapy device;
receive commands or instructions to establish a new exercise or therapy routine with specific parameters or measurements;
capture and record parameters and measurements for the new exercise and therapy routine that are transmitted from the exercise or therapy device while the user is performing the new exercise or therapy routine;
process the captured recorded parameters and measurements to calculate ranges of parameters and measurements for the new exercise or therapy routine; and
display the ranges of parameters and measurements for the new exercise or the therapy routine.

13. The method of claim 12, wherein the computer-readable instructions are executable by the one or more processors to:
receive changes to the ranges of parameters and measurements for the new exercise or the therapy routine; and
store the changed ranges of parameters and measurements for the new exercise or therapy routine.

14. The method of claim 12, wherein the computer-readable instructions are executable by the one or more processors to:
receive a name identifier for the new exercise or therapy routine; and
store the name identifier and the ranges of parameters and measurements for the name identifier.

15. The method of claim 12, wherein the parameters and measurements comprise multi-axis angular measurements from the one or more accelerometers, gyroscopes or directional sensors; magnetometers; receiving grip strength measurements from the one or more pressure sensors or strain gauges; velocity or acceleration measurements from the one or more accelerometers; location measurements for the one or more gyroscopes or direction sensors; or weight lifted measurements from the one or more pressure sensors or strain gauges.

16. A method of utilizing an intelligent exercise device in performing an exercise, comprising:
one or more processors;
one or more memory devices;
computer-readable instructions stored in the one or more memory devices, the computer- readable instructions accessible from the one or more memory devices and executable by the one or more processors to:
establishing a wireless communication connection with a computing device, the computing device being separate from an exercise or therapy device;
receive a selection of an exercise or therapy device;
receive a selection of an exercise or therapy routine;
initiate the exercise or therapy routine;
receive parameters or measurements from the intelligent exercise or therapy device as the user is performing the exercise or therapy routine; and
display the received parameters or measurements from the intelligent exercise or therapy device utilizing at least a two-dimensional graph on a monitor, wherein the parameters or measurements include multi-axis angular measurements from the one or more accelerometers, gyroscopes or directional sensors, magnetometers; receiving grip strength measurements from the one or more pressure sensors or strain gauges; velocity or acceleration measurements from the one or more accelerometers; location measurements for the one or more gyroscopes or direction sensors; applied load or weight lifted measurements from the one or more pressure sensors or strain gauges.

17. The method of claim 16, further comprising computer-readable instructions executable by the one or more processors to:
record the received parameters or measurements for the exercise or therapy routine along with a name identifier of the user and a routine identifier of the exercise or therapy routine.

18. The method of claim 16, further comprising computer-readable instructions executable by the one or more processors to:
compare the received parameters or measurements to stored ranges of parameters for the selected exercise routine; and
generate a message or notification that the user is performing outside of the stored ranges of parameters for the selected exercise routine.

19. The method of claim 18, further comprising computer-readable instructions executable by the one or more processors to:
receive new ranges of parameters or measurements for the selected exercise or therapy routine from the user; and
store the new ranges of parameters or measurements for the selected exercise or therapy routine and the name identifier in the one or more memory devices.

20. The method of claim 16, further comprising computer-readable instructions executable by the one or more processors to:
perform the steps of claim 16 for multiple exercise or therapy routines; and
generate performance statistic measurements for the user, the performance statistic measurements including repetition count, exercise or therapy accuracy, or exercise program completion levels;
display the performance statistic measurements; and
communicate the performance statistic measurements to the user and/or a medical or exercise professional.
